(19) United States
(12) Patent Application Publication
(10) Pub. No.: US 2023/0381261 A1
(43) Pub. Date: Nov. 30, 2023

(54) A Purified Extract Isolated from Agrimonia Coreana Nakai Containing Abundant Amount of Active Ingredient, the Preparation Thereof, the Composition Comprising the Same as an Active Ingredient for Preventing or Treating Inflammation, Allergy and Atopic Dermatitis and the Use Thereof

(71) Applicant: Yuran NAM, Goyang-si (KR)

(72) Inventors: Woo Kyung Kim, Goyang-si (KR); Joo Hyun Nam, Seoul (KR)

(21) Appl. No.: 18/032,023

(22) PCT Filed: Oct. 12, 2021
(86) PCT No.: PCT/KR2021/014020

§ 371 Date: Apr. 14, 2023

(30) Foreign Application Priority Data
Oct. 19, 2020 (KR) 10-2020-0135543
May 12, 2021 (KR) 10-2021-0061186

(51) Int. Cl. A61K 36/73; A61K 31/7048; A61K 31/352; A61P 29/00; A61P 37/08

(52) U.S. Cl. CPC A61K 36/73; A61K 31/7048; A61K 31/352; A61P 29/00; A61P 37/08

(57) ABSTRACT

The present invention relates to a purified extract isolated from Agrimonia coreana NAKAI containing abundant amount of active ingredient, the preparation thereof, the composition comprising the same as an active ingredient for preventing or treating inflammation, allergy and atopic dermatitis and the use thereof. The present invention provides novel industrialized method for preparing purified extract (APK200608) from Agrimonia coreana NAKAI, a Korean native plant, which can provide more abundant active ingredients showing potent ant-inflammatory activity comparing with the crude extract prepared by the conventional method disclosed in the prior art or previous known extract of Agrimonia pilosa.

### TECHNICAL FIELD

The present invention is related to a purified extract isolated from Agrimonia coreana NAKAI containing abundant amount of active ingredient, the preparation thereof, the composition comprising the same as an active ingredient for preventing or treating inflammation, allergy and atopic dermatitis and the use thereof.

### BACKGROUND ART

Chronic recurrent dermatological inflammatory diseases such as atopic dermatitis (atopic dermatitis) and psoriasis are very difficult to treat because of the wide variety of causes.

In general, the prevalence of atopic dermatitis has been higher in industrialized countries, with 10-20% in children and 1-3% in adults, but has recently been similar worldwide. Atopic dermatitis is characterized by severe fecundity and dry skin, and may be accompanied by other allergic diseases such as asthma and allergic rhinitis (J Korean Med Assoc 2020; 63 (5) : 251-258.)

Currently, immunosuppressants are commonly used for treatment of atopic dermatitis, and steroids as well as calcinurin inhibitors are used as typical immunosuppressants. Histamines secreted from the mast cell causes itching and the itching has been treated in combination with antihistamines to control the symptoms (Allergy Asthma Respir Dis 5(5):248-255, September 2017).

Steroids commonly used in atopic dermatitis inhibit the production of IL-1α, an inflammatory cytokine, and inhibit IL-2 secreted by T cells, thereby inhibiting T cell proliferation and inducing apoptosis. (J Korean Med Assoc 2020; 63 (5) : 251-258).

Calcineurin inhibitors among the immunosuppressants used in atopic dermatitis, has main mechanisms for inhibiting the activity of T helper cells.

T cells activated by antigen begin immune response activation, starting with an increase in intracellular calcium concentration through the ORAI1 ion pathway, and the influx of intracellular calcium binds to calmodulin to activate calcinulin. Calcineurin activates NFAT among the transcriptional regulators to induce inflammatory cytokine reproduction (J Korean Med Assoc 2020; 63 (5) : 251-258).

At present, various antihistamine or steroids such as an injection and ointment form of cortisol, prednisolone, methylprednisolone, dexamethasone etc, are commonly used for the treatment of atopic dermatitis, however, it dosed not provides with satisfactory efficacy.

In particular, the therapy with steroids induce more severe hypersensitivity due to the enlarged capillaries and thinned skin layers, and furthermore, if steroid application is suspended, resulting steroid rebound causes to show more severe symptom for example, Cushing syndrome, edema, diabetes, osteoporosis, growth disorders in children etc.

Calcineurin inhibitors as well as TH2 Cytokine Inhibitor, representative treating agents for the treatment of atopic dermatitis, affect not only T cells but also all cells of the human body since they have no specificity for T cells, and they have been known to show various side effects, such as fatal side effects on kidneys or liver, occurrence of many types of cancer.

Accordingly, there has been still needed to develop more effective treating agent in treating and alleviating inflammatory diseases such as atopic dermatitis from natural source with low side effects than conventionally used drugs till now.

Agrimonia pilosa is a perennial herbaceous species of the double-leaf plant rose family, distributed throughout Korea, as well as eastern Russia, Mongolia, Europe, Okinawa, Japan, and northeastern China. Agrimonia pilosa is distinguished from Agrimonia coreana NAKAI because it usually does not cover the stem in the shape of a chin-leaf sickle, whereas Agrimonia coreana NAKAI wraps around the stem in the shape of a semicircular or fan. Also, the flowers of Agrimonia pilosa are closely attached to the firing sequence, distinguishing them from whereas Agrimonia coreana NAKAI, which are somewhat sloppy (Korean Bio Resource Information System, https://www.bris.go.kr)

Agrimonia coreana NAKAI, a Korean native plant, is a perennial herbaceous species of the rose family, native to South Korea, and has been reported to contain agrimonin, agrimonolide, tannn, saponin etc and agrimonin, among them has been reported to show blood coagulating activity, hypoglycemic activity, anti-pesticide activity etc (Chung B. S et al, Dohaehyangyakdaesajeon, youngrimsa, 2^{nd} Ed. P636-637, 1998).

Korea Patent Publication No. 10-2010-0128770 discloses on “cosmetic composition containing water/ethanol extract of Agrimonia pilosa showing anti-atopic dermatitis”; Korea Patent Publication No. 10-2012-0126416 discloses on “Composition comprising extract of Agrimonia pilosa having treating or preventing inflammation, allergy or asthma disease”; Korea Patent Publication No. 10-2012-0003317 discloses on “ Composition comprising extract of Agrimonia pilosa for inhibiting the degranulation of mast cell”; Korea Patent Publication No. 2001-0053978 discloses on “anti-irritant cosmetic composition comprising extract of Agrimonia pilosa”: Korea Patent Registration No. 10-1554030 discloses on “cosmetic composition comprising a root extract of Agrimonia pilosa for improving skin trouble”etc.

There have been several literatures on the anti-inflammatory compounds which has been isolated from Agrimonia pilosaa, for examples,
(a)Luteolin; Kim J J, Jiang J, Shim D W, Kwon S C, Kim T J, Ye S K, Kim M K, Shin Y K, Koppula S, Kang T B, Choi D K, Lee K H. (2012) Anti-inflammatory and anti-allergic effects of Agrimonia pilosa Ledeb extract on murine cell lines and OVA-induced airway inflammation. J. Ethnopharmacol. 140(2):213-221); Apigenin [Aziz N, Kim M Y, Cho J Y.(2018) Anti-inflammatory effects of luteolin: A review of in vitro, in vivo, and in silico studies. J Ethnopharmacol. 28; 225:342-358.],
(b)Kaempferol [Pang L, Zou S, Shi Y, Mao Q, Chen Y. (2019) Apigenin attenuates PM2.5-induced airway hyperresponsiveness and inflammation by down-regulating NF-_{K}B in murine model of asthma Int J Clin Exp Pathol. 12(10):3700-3709],
(c)Quercetin [Alam W, Khan H, Shah M A, Cauli O, Saso L.(2020) Kaempferol as a Dietary Anti-Inflammatory Agent: Current Therapeutic Standing. Molecules 7;25(18):E4073; Guiling Chen, Yang Ye, Ming Cheng, Yi Tao, Kejun Zhang, Qiong Huang, Jingwen Deng, Danni Yao, Chuanjian Lul and Yu Huang. Quercetin Combined With Human Umbilical Cord Mesenchymal Stem Cells Regulated Tumour Necrosis Factor-a/Interferon-g-Stimulated Peripheral Blood Mononuclear Cells via Activation of Toll-Like Receptor 3 Signalling. Front Pharmacol. 2020; 11: 499.],
(d)Luteolun 7-glucuronide [Young-Chang Cho, Jiyoung Park, Sayeon Cho (2020) Anti-Inflammatory and Anti-Oxidative Effects of luteolin-7-O-glucuronide in LPS-Stimulated Murine Macrophages through TAK1 Inhibition and Nrf2 Activation. Int J Mol Sci. 16;21(6):2007],
(e)Apigenin 7-glucuronide [Weicheng Hu, Xinfeng Wang, Lei Wu, Ting Shen, Lilian Ji, Xihong Zhao, Chuan-Ling Si, Yunyao Jiang, Gongcheng Wang (2016); Apigenin-7-O-β-D-glucuronide inhibits LPS-induced inflammation through the inactivation of AP-1 and MAPK signaling pathways in RAW 264.7 macrophages and protects mice against endotoxin shock. Food Funct. 7(2):1002-13.

However, there has been not reported or disclosed about the efficient method for preparing more potent and the inventive purified extract isolated from the extract containing more abundant ingredients, or the compounds of Agrimonia coreana NAKAI, a Korean native plant, showing anti-inflammatory, anti-allergy and anti-atopic dermatitis activity than those in the above cited literatures, the disclosures of which are incorporated herein by reference.

Furthermore, there have been difficulties in mass-production and industrialization using by the extract of Agrimonia pilosa, since the plant extract contains very little anti-inflammatory ingredients such as luteolun 7-glucuronide, apigenin 7-glucuronide, luteolin, apigenin, kaempferol, quercetin etc.

Based on the previous studies on the anti-inflammatory and anti-allergy activity of the extract of Agrimonia pilosa, disclosed in the above cited literatures, the present inventors have tried to develop more efficient method for preparing more potent and more abundant ingredients showing anti-inflammatory, anti-allergy and anti-atopic dermatitis activity isolated from the extract of Agrimonia coreana NAKAI, a Korean native plant, till now.

Accordingly, the present inventors have found the novel industrialized method for preparing novel purified extract containing more abundant active ingredients for example, newly found alphitolic acid as well as various flavonoid derivatives such as luteolun 7-glucuronide, apigenin 7-glucuronide, luteolin, apigenin, kaempferol, quercetin etc from the extract of Agrimonia coreana NAKAI, a Korean native plant, and the purified extract showed more potent anti-inflammatory, anti-allergy and anti-atopic dermatitis activity than that prepared by the conventional preparation method disclosed in the prior art through various tests such inhibition effect on T cell proliferation (Experimental Example 1), inhibition effect on IL-2 release in Jurkat T cell (Experimental Example 2), inhibition effect on degranulation in mast cell (Experimental Example 3), inhibition effect on ORAI1 ion channel regulating calcium signaling in cell (Experimental Example 4),.

### DISCLOSURE OF INVENTION

### Technical Problem

To investigate the anti-inflammatory effect of novel purified extract containing more abundant active ingredients for example, newly found alphitolic acid as well as various flavonoid derivatives such as luteolun 7-glucuronide, apigenin 7-glucuronide, luteolin, apigenin, kaempferol, quercetin etc from the extract of Agrimonia coreana NAKAI, a Korean native plant, rather than the previous known extract of Agrimonia pilosa, the inventors of present invention have intensively carried out various experiments including such. HPLC analysis of inventive purified extract (APK200608) isolated from Agrimonia coreana NAKAI, a Korean native plant, as well as the previous known extract of Agrimonia pilosa, (Comparative Example and Examples); inhibition effect on T cell proliferation (Experimental Example 1), inhibition effect on IL-2 release in Jurkat T cell (Experimental Example 2), inhibition effect on degranulation in mast cell (Experimental Example 3), inhibition effect on ORAI1 ion channel regulating calcium signaling in cell (Experimental Example 4).

As a result of these investigations, the inventors finally completed the present invention by confirming that inventive novel purified extract from the extract of Agrimonia coreana NAKAI, a Korean native plant, contains more abundant active ingredients for example, newly found alphitolic acid as well as various flavonoid derivatives such as luteolun 7-glucuronide, apigenin 7-glucuronide, luteolin, apigenin, kaempferol, quercetin etc, in particular, luteolin (4.86 times) and apigenin (7.3 times), than the previous known extract of Agrimonia pilosa, and furthermore, the inventive novel purified extract from the extract of Agrimonia coreana NAKAI, a Korean native plant, showed un-expectedly more-potent treating or inhibiting effect on inflammation, allergy and atopic dermatitis diseases than the previous known extract of Agrimonia pilosa.

### Solution to Problem

The technical solution to solve the problem of the background art is for the development of novel industrialized method for preparing novel purified extract containing more abundant active ingredients showing anti-inflammatory activity for example, newly found alphitolic acid as well as various flavonoid derivatives such as luteolun 7-glucuronide, apigenin 7-glucuronide, luteolin, apigenin, kaempferol, quercetin etc, from the extract of Agrimonia coreana NAKAI, a Korean native plant.

The present invention provides a novel method for preparing purified extract containing abundant active ingredients showing anti-inflammatory activity for example, newly found alphitolic acid as well as various flavonoid derivatives such as luteolun 7-glucuronide, apigenin 7-glucuronide, luteolin, apigenin, kaempferol, quercetin etc, from the extract of Agrimonia coreana NAKAI, a Korean native plant, and the extract prepared thereby.

The present invention also provides a pharmaceutical composition and a health food comprising the novel purified extract containing abundant active ingredients showing anti-inflammatory activity for example, newly found alphitolic acid as well as various flavonoid derivatives such as luteolun 7-glucuronide, apigenin 7-glucuronide, luteolin, apigenin, kaempferol, quercetin etc, from the extract of Agrimonia coreana NAKAI, a Korean native plant, as an active ingredient in an effective amount to treat and prevent inflammatory, allergic or atopic dermatitis disease.

The present invention also provides a use of novel purified extract containing abundant active ingredients showing anti-inflammatory activity for example, newly found alphitolic acid as well as various flavonoid derivatives such as luteolun 7-glucuronide, apigenin 7-glucuronide, luteolin, apigenin, kaempferol, quercetin etc, from the extract of Agrimonia coreana NAKAI, a Korean native plant, showing anti-inflammatory, anti-allergic and anti-atopic activity.

The present invention also provides a method of treating or preventing inflammatory, allergic or atopic dermatitis disease in a mammal comprising administering to said mammal an effective amount of novel purified extract containing abundant active ingredients showing anti-inflammatory activity for example, newly found alphitolic acid as well as various flavonoid derivatives such as luteolun 7-glucuronide, apigenin 7-glucuronide, luteolin, apigenin, kaempferol, quercetin etc, from the extract of Agrimonia coreana NAKAI, a Korean native plant, together with a pharmaceutically acceptable carrier thereof.

Accordingly, it is an object of the present invention to provide novel purified extract (APK200608) containing abundant active ingredients showing anti-inflammatory activity for example, newly found alphitolic acid as well as various flavonoid derivatives such as luteolun 7-glucuronide, apigenin 7-glucuronide, luteolin, apigenin, kaempferol, quercetin etc from the extract of Agrimonia coreana NAKAI, a Korean native plant.

The term “flavonoid derivatives” disclosed herein comprises luteolun 7-glucuronide, apigenin 7-glucuronide, luteolin, apigenin, kaempferol, quercetin etc.

The term “Agrimonia coreana NAKAI” disclosed herein comprises the aerial part, whole plant, leaf, stem, or root of cultivated or naturally grown plant located in Chung-cheong do province, Jeolla do province, Gyeongsang do province etc in South Korea and commercially available plant, but not intent to limit thereto herein.

The term “novel purified extract” disclosed herein comprises the purified extract fractionated with HP20 column from the extract of Agrimonia coreana NAKAI, a Korean native plant, showing anti-inflammatory, anti-allergic and anti-atopic activity and containing abundant active ingredients showing anti-inflammatory activity for example, newly found alphitolic acid as well as various flavonoid derivatives such as luteolun 7-glucuronide, apigenin 7-glucuronide, luteolin, apigenin, kaempferol, quercetin etc (designated as “APK200608” hereinafter).

Specifically, the term “novel purified extract from the extract of Agrimonia coreana NAKAI (APK200608))” is characterized by containing 0.5-1.5 weight part (w/w) luteolun 7-glucuronide, 0.5-1.8 weight part (w/w) apigenin 7-glucuronide, 0.005-0.015 weight part (w/w) luteolin, 0.0050-0.0090 weight part (w/w) apigenin, 0.0010-0.0030 weight part (w/w) kaempferol, and 0.0040-0.0098 weight part (w/w) quercetin based on the weight of total extract (100%) of Agrimonia coreana NAKAI, preferably, 0.8-1.3 weight part (w/w) luteolun 7-glucuronide, 0.8-1.6 weight part (w/w) apigenin 7-glucuronide, 0.008-0.012 weight part (w/w) luteolin, 0.0060-0.0080 weight part (w/w) apigenin, 0.0015-0.0025 weight part (w/w) kaempferol, and 0.0055-0.0095 weight part (w/w) quercetin based on the weight of total extract (100%) of Agrimonia coreana NAKAI, more preferably, 0.9-1.2 weight part (w/w) luteolun 7-glucuronide, 1.0-1.4 weight part (w/w) apigenin 7-glucuronide, 0.009-0.011 weight part (w/w) luteolin, 0.0065-0.0075 weight part (w/w) apigenin, 0.0018-0.0023 weight part (w/w) kaempferol, and 0.0065-0.0085 weight part (w/w) quercetin based on the weight of total extract (100%) of Agrimonia coreana NAKAI, or characterized by having the relative mixed weight ratio (w/w) between the weight of each flavonoid derivative, of 400.0-650.0 (w/w) luteolun 7-glucuronide, 550.0-750.0 (w/w) apigenin 7-glucuronide, 2.0-10.0 (w/w) luteolin, 1.5-7.0 (w/w) apigenin, 1 (w/w) kaempferol, and 1.5-7.5 (w/w) quercetin; preferably, 450.0-600.0 (w/w) luteolun 7-glucuronide, 600.0-700.0 (w/w) apigenin 7-glucuronide, 2.5-8.0 (w/w) luteolin, 2.0-5.0 (w/w) apigenin, 1 (w/w) kaempferol, and 2.5-5.5 (w/w) quercetin; more preferably, 480.0-550.0 (w/w) luteolun 7-glucuronide, 620.0-670.0 (w/w) apigenin 7-glucuronide, 3.5-6.0 (w/w) luteolin, 3.0-4.0 (w/w) apigenin, 1 (w/w) kaempferol, and 3.0-4.5 (w/w) quercetin.

More specifically, the term “novel purified extract from the extract of Agrimonia coreana NAKAI (APK200608)” is characterized by being prepared by the process of; adding at least one extracting solvent selected from water, spirit, C1-C4 lower alcohol such as methanol, ethanol, butanol etc or the mixtures thereof, preferably, mixture of water and ethanol or spirit , more preferably, 30-80(w/w) ethanol or spirit in water to the dried aerial part, whole plant, leaf, stem, or root of cultivated or naturally grown of Agrimonia coreana NAKAI, a Korean native plant, located in Chung-cheong do province, Jeolla do province, Gyeongsang do province etc at the 1st step; subjecting to at least one extraction method selected from reflux extraction with hot water, cold water extraction, ultra-sonication or conventional extraction, preferably reflux extraction at the temperature ranging from 10 to 100° C., preferably from 20 to 90° C., for the period ranging from 30 mins to 72 hours, preferably, 3 to 72 hours, repeatedly, 1-20 times, preferably, 2-10 times, to afford the 1st extract at 2nd step; and subjecting the 1st extract to at least one purification process selected from the group consisting of reverse phase partition chromatography, normal phase partition chromatography, ion exchange chromatography, and size exclusion chromatography to afford the novel purified extract from the extract of Agrimonia coreana NAKAI (APK200608) containing 0.5-1.5 weight part (w/w) luteolun 7-glucuronide, 0.5-1.8 weight part (w/w) apigenin 7-glucuronide, 0.005-0.015 weight part (w/w) luteolin, 0.0050-0.0090 weight part (w/w) apigenin, 0.0010-0.0030 weight part (w/w) kaempferol, and 0.0040-0.0098 weight part (w/w) quercetin based on the weight of total extract (100%) of Agrimonia coreana NAKAI, preferably, 0.8-1.3 weight part (w/w) luteolun 7-glucuronide, 0.8-1.6 weight part (w/w) apigenin 7-glucuronide, 0.008-0.012 weight part (w/w) luteolin, 0.0060-0.0080 weight part (w/w) apigenin, 0.0015-0.0025 weight part (w/w) kaempferol, and 0.0055-0.0095 weight part (w/w) quercetin based on the weight of total extract (100%) of Agrimonia coreana NAKAI, more preferably, 0.9-1.2 weight part (w/w) luteolun 7-glucuronide, 1.0-1.4 weight part (w/w) apigenin 7-glucuronide, 0.009-0.011 weight part (w/w) luteolin, 0.0065-0.0075 weight part (w/w) apigenin, 0.0018-0.0023 weight part (w/w) kaempferol, and 0.0065-0.0085 weight part (w/w) quercetin based on the weight of total extract (100%) of Agrimonia coreana NAKAI, or characterized by having the relative mixed weight ratio (w/w) between the weight of each flavonoid derivative, of 400.0-650.0 (w/w) luteolun 7-glucuronide, 550.0-750.0 (w/w) apigenin 7-glucuronide, 2.0-10.0 (w/w) luteolin, 1.5-7.0 (w/w) apigenin, 1 (w/w) kaempferol, and 1.5-7.5 (w/w) quercetin; preferably, 450.0-600.0 (w/w) luteolun 7-glucuronide, 600.0-700.0 (w/w) apigenin 7-glucuronide, 2.5-8.0 (w/w) luteolin, 2.0-5.0 (w/w) apigenin, 1 (w/w) kaempferol, and 2.5-5.5 (w/w) quercetin; more preferably, 480.0-550.0 (w/w) luteolun 7-glucuronide, 620.0-670.0 (w/w) apigenin 7-glucuronide, 3.5-6.0 (w/w) luteolin, 3.0-4.0 (w/w) apigenin, 1 (w/w) kaempferol, and 3.0-4.5 (w/w) quercetin.

to treat and prevent inflammatory, allergic or atopic dermatitis disease.

Accordingly, in an another embodiment of the present invention, the present invention also provides a method for preparing novel purified extract from the extract of Agrimonia coreana NAKAI (APK200608) comprising the steps of; adding at least one extracting solvent selected from water, spirit, C1-C4 lower alcohol such as methanol, ethanol, butanol etc or the mixtures thereof, preferably, mixture of water and ethanol or spirit , more preferably, 30-80(w/w) ethanol or spirit in water to the dried aerial part, whole plant, leaf, stem, or root of cultivated or naturally grown of Agrimonia coreana NAKAI, a Korean native plant, located in Chung-cheong do province, Jeolla do province, Gyeongsang do province etc at the 1st step; subjecting to at least one extraction method selected from reflux extraction with hot water, cold water extraction, ultra-sonication or conventional extraction, preferably reflux extraction at the temperature ranging from 10 to 100° C., preferably from 20 to 90° C., for the period ranging from 30 mins to 72 hours, preferably, 3 to 72 hours, repeatedly, 1-20 times, preferably, 2-10 times, to afford the 1st extract at 2nd step; and subjecting the 1st extract to at least one further purification process selected from the group consisting of reverse phase partition chromatography, normal phase partition chromatography, ion exchange chromatography, and size exclusion chromatography to afford the novel purified extract from the extract of Agrimonia coreana NAKAI (APK200608) containing 0.5-1.5 weight part (w/w) luteolun 7-glucuronide, 0.5-1.8 weight part (w/w) apigenin 7-glucuronide, 0.005-0.015 weight part (w/w) luteolin, 0.0050-0.0090 weight part (w/w) apigenin, 0.0010-0.0030 weight part (w/w) kaempferol, and 0.0040-0.0098 weight part (w/w) quercetin based on the weight of total extract (100%) of Agrimonia coreana NAKAI to treat and prevent inflammatory, allergic or atopic dermatitis disease.

Specifically, the term “further purification process” is selected from (i) reverse phase partition chromatography, (ii) normal phase partition chromatography, (iii) ion exchange chromatography or (iv) size exclusion chromatography, preferably, reverse phase partition chromatography or any chromatography using by any resin as a stationary phase which can retain non-polar substance while eluting polar substance, for example, Sephadex resin such as Sephadex, Sephadex LH20, Sephadex G-25, Sephadex G-10, Sepharose, Superdex, methylacrylate resin, carboxymethyl cellulose, sulphopropyl cellulose, carboxymethyl Sephadex, sulphopropyl Sephadex, carboxymethyl Sepharose, sulphopropyl Sepharose and the like; reverse polymer resin using by Stylene-divinylbenzen co-polymer such as Polymer X, HP20, PRP-h1 Polymer and the like or Methacrylate support resin etc; normal Silica gel such as BPC (Bonded pahse chromatography) product, Silica product procured from YMC Co. Ltd, Silica product procured from DAISO Co. Ltd, Silica product procured from ASAHI Co. Ltd, Silica product procured from COSMOSYL Co. Ltd and the like; ODS product used for HPLC filler such as ODS product procured from YMC Co. Ltd, ODS product procured from DAISO Co. Ltd, ODS product procured from ASAHI Co. Ltd, ODS product procured from CHEMCO Co. Ltd, ODS product procured from Merck Co. Ltd ODS product procured from COSMOSYL Co. Ltd ODS product procured from FUJI Co. Ltd and the like.

In a preferred embodiment adopting (i) reverse phase partition chromatography as a further purification process of the present invention, the “stationary phase in the above-described reverse phase partition chromatography” may be any stationary phases such as reverse phase substance as a stationary phase which can retain non-polar substance while eluting polar substance, preferably, Silica gel based stationary phase, polymer based stationary phase such as polystyrene etc and the like, more preferably, Silica gel derivatives such as C2, C4, C6, C8, C10, C12, 14, C16, C18 and the like; or a polymer based stationary phase such as PS-2, Oasis HLB and the like, more and more preferably, reverse phase Silica gel (C18(IV)-D), ODS-A/ODS-AQ product from YMC Co. Ltd., SP-C-ODS product from CHEMCO Co. Ltd., SP-ODS-RPS product from DAISO Co. Ltd., 5C18 product from COSMOSYL Co. Ltd., Chromatorex product from FUJI Co. Ltd., etc.

In a preferred embodiment adopting (i) reverse phase partition chromatography as a further purification process of the present invention, the “mobile phase in the above-described (i) reverse phase partition chromatography”may be at least one solvent selected from water, acetonitrile, lower alcohol such as methanol, ethanol, butanol etc, tetrahydrofuran (THF) or the mixture thereof, preferably, water, lower alcohol such as methanol, ethanol, butanol etc, or the mixture thereof, more preferably, the mixture solvent of water and methanol, more and more preferably, the mixture solvent of water and methanol with starting from 90:10(v/v) to 60:40(v/v) to elute polar substance.

In a preferred embodiment adopting (ii) normal phase partition chromatography as a further purification process of the present invention, the “stationary phase in the above-described normal phase partition chromatography”may be any stationary phases such as normal phase substance as a stationary phase which can retain polar substance while eluting non-polar substance, preferably, Silica gel, Fluorosyl, or alumina based stationary phase, CN, Diol, or NH2 moiey polymer based stationary phase and the like, more preferably, Silica gel, Fluorosyl, or alumina based stationary phase, etc.

In a preferred embodiment adopting (ii) normal phase partition chromatography as a further purification process of the present invention, the “mobile phase in the above-described (ii) normal phase partition chromatography”may be at least one solvent selected from hexane, heptane, ethylacetate, ethanol, diethylether, 2-propanol or the mixture thereof, preferably, hexane, heptane, ethylacetate or the mixture thereof to elute non-polar substance.

In a preferred embodiment adopting (iii) ion exchange chromatography as a further purification process of the present invention, the “stationary phase in the above-described (iii) ion exchange chromatography”may be any high molecular stationary phases as a stationary phase which have charged cross-linking moiety, preferably, cation exchange resin, anion exchange resin, or synthetic adsorbent, and the like, more preferably, strongly acidic cation exchange resin such as AG 50W-x8, Amberlite IR-120, Dowex 60W-x8, SKIB etc; weakly acidic cation exchange resin such as Amberlite IRA-67, Dowex 3-x4A etc; strongly basic cation exchange resin such as DIAION SKIB, DIAION PK216, DIAION CR20, DIAION UBK555 (Mitsubishi Chemical Co.), TRILITE SPC 160H, TRILITE SPC 180H, TRILITE SPC 400JH (Samyang Co. Ltd.), AMBERLITE 200C Na, AMBERLITE CG50, AMBERLITE CR1310 Na, AMBERJET 200H, AMBERLYST 131 WET, ALBERLYST 232 WET (ROHM and HAAS Co. Ltd.), Lewatit VP OC 1800, Lewatit VP OC 1812, Lewatit MDS1368 Na, Lewaitit K1221 (Bayer Co. Ltd.), PUROLITE PCR833CA, PUROLITE C145 (Purolite Co. Ltd.), MFG210, MFG 250 (Finex Co. Ltd.) etc; strongly basic anion exchange resin such as SA11A, SA20A, SA21A etc; or CaptoQ (GE Healthcare Co. Ltd.), or the resin having similar property thereto such as Toyopearl QEA (Tosoh Co. Ltd.), Q Sepharose FF (GE Healthcare Co. Ltd.), Fractogel EMD, Fractogel TMAE, Fractogel HICAP (Merck KGaA Co. Ltd or Darmstadt Co. Ltd.); more and more preferably, SA21A; adsorbent such as SP207, HP20SS, HP20 etc, more preferably, HP 20.

In a preferred embodiment adopting (iv) size exclusion chromatography as a further purification process of the present invention, the “stationary phase in the above-described (iv) size exclusion chromatography” may be any gel type stationary phases as a stationary phase which can separate by the size of sample, preferably, dextran-based gel such as sephadex (for example, sephadex G-25), polyacrylamide-based gel such as Sephacryl (for example, Sephacryl-S400), Agarose-based gel such as Superose or Sepharose (for example, Sepharose CL-4B) or the combinations thereof such as Superdex 200 combination Dextran (For example, Sephadex™), or cross-linked Agarose gel (Superose™) and the like, however it shall be not limited thereto herein. The “mobile phase in the above-described (iv) size exclusion chromatography” may be buffer solution selected from the group consisting of sodium acetate buffer, sodium phosphate buffer, ammonium acetate buffer, MES (2-(N-morpholino)ethanesulphonic acid), Bis-Tris[2-Bis(2-hydroxyethyl)amino-2-(hydroxymethyl)-1,3-propandiol], ADA [N-(2-acetamido)iminodiacetate), PIPES [piperazine-N,N-Bis(2-ethanesulophonic acid)], BES [N.N′-Bis(2-hydroxyethyl)-2-aminoethansulphonic acid), MOPS [3-(N-morpholino)propansulphonic acid)], TES (N-Tris(hydroxymethyl)methyl-2-aminoethanesulphonic acid], HEPES [N-2-hydroxyethyl-piperazine-N′-2-ethanesulphonic acid), and the like; preferably, sodium acetate buffer, sodium phosphate buffer, or ammonium acetate buffer.

In a preferred embodiment of the present invention, the present invention can also perform (v) Gel permeation chromatography or Gel filtration chromatography in addition to (i) reverse phase partition chromatography, (ii) normal phase partition chromatography, (iii) ion exchange chromatography, (iv) size exclusion chromatography or the combination thereof, as a further purification process disclosed herein.

The present invention also provides novel purified extract from the extract of Agrimonia coreana NAKAI (APK200608) prepared by the above-described preparation methods.

The present invention also novel purified extract from the extract of Agrimonia coreana NAKAI (APK200608) prepared by the above-described preparation methods, which contains 0.5-1.5 weight part (w/w) luteolun 7-glucuronide, 0.5-1.8 weight part (w/w) apigenin 7-glucuronide, 0.005-0.015 weight part (w/w) luteolin, 0.0050-0.0090 weight part (w/w) apigenin, 0.0010-0.0030 weight part (w/w) kaempferol, and 0.0040-0.0098 weight part (w/w) quercetin based on the weight of total extract (100%) of Agrimonia coreana NAKA Ito treat and prevent inflammatory, allergic or atopic dermatitis disease.

The term “purified extract” disclosed herein may be used as a dried form prepared by the vacuum evaporation method, freeze dry method or hot-air drying method etc.

The term “inflammatory disease” disclosed herein comprises eczema, conjunctivitis, periodontal disease, rhinitis, otitis media, laryngopharyngitis, tonsillitis, pneumonia, gastric ulcer, gastritis, Crohn's disease, colitis, hemorrhoid, gout, ankylosing spondylitis, rheumatic fever, systemic lupus erythematosus, fibromyalgia, psoriatic arthritis, osteoarthritis, rheumatic arthritis, periarthritis of shoulder, tendinitis, tenosynovitis, peritendinitis, myositis, hepatitis, cystitis, nephritis, Sjogren's syndrome, multiple sclerosis, chronic inflammatory disease, acute inflammatory disease etc, but not intended herein to limit thereto, preferably, eczema, rheumatic arthritis, chronic inflammatory disease, acute inflammatory disease etc,

The term “allergic disease” disclosed herein comprises allergic rhinitis, allergic dermatitis, contact dermatitis, hives, insect allergy, food allergy, drug allergy, allergic conjunctivitis, hypersensitivity etc, but not intended herein to limit thereto, preferably, allergic rhinitis, allergic dermatitis, contact dermatitis, hives, insect allergy, food allergy, drug allergy, more preferably, allergic dermatitis, contact dermatitis.

The term“prevent” disclosed herein comprises any act to inhibit or postpone the occurrence of certain disease or disorder disclosed herein by way of administrating the inventive composition; and the term “treat”disclosed herein comprises any act to alleviate or favorably changing the symptom associated with certain disease or disorder disclosed herein by way of administrating the inventive composition.

The present inventors have found that the novel industrialized method for preparing purified extract (APK200608) from Agrimonia coreana NAKAI, a Korean native plant, can provide more abundant active ingredients showing potent ant-inflammatory activity, for example, newly found alphitolic acid (Table 6 and FIG. 6) as well as various flavonoid derivatives such as luteolun 7-glucuronide (10.480 μg/mg), apigenin 7-glucuronide (13.546 μg/mg), luteolin (0.102 μg/mg), apigenin (0.073 μg/mg), kaempferol (0.021 μg/mg) and quercetin (0.079 μg/mg) based on total extract (1 mg) (Table 4 and FIG. 4), in particular, luteolin (4.86 times) and apigenin (7.3 times), comparing with the crude extract prepared by the conventional method disclosed in the prior art or previous known extract of Agrimonia pilosa which does not contain alphitoic acid and contains luteolun 7-glucuronide (5.110-6.872 μg/mg), apigenin 7-glucuronide (6.691-9.428 μg/mg), luteolin (0.021-0.046 μg/mg), apigenin (0.010-0.028 μg/mg), kaempferol (0-0.009 μg/mg) and quercetin (0.042-0.058 μg/mg) based on total extract (1 mg) (Table 2 and FIG. 1,2) of Agrimonia coreana NAKAI; as well as the inventive purified extract showed more potent anti-inflammatory, anti-allergy and anti-atopic activity than that prepared by the conventional preparation method through various in test such as inhibition effect on T cell proliferation (Experimental Example 1), inhibition effect on IL-2 release in Jurkat T cell (Experimental Example 2), inhibition effect on degranulation in mast cell (Experimental Example 3), inhibition effect on ORAI1 ion channel regulating calcium signaling in cell (Experimental Example 4), therefore, it is confirmed that inventive purified extract is very useful in the alleviation or treatment of inflammatory disease, allergic disease and atopic dermatitis disease.

Accordingly, in accordance with the other aspect of the present invention, present invention provide a pharmaceutical composition comprising novel purified extract from the extract of Agrimonia coreana NAKAI (APK200608) as an active ingredient for the treatment and prevention of inflammatory, allergic or atopic dermatitis disease.

Present invention provide a pharmaceutical composition comprising novel purified extract from the extract of Agrimonia coreana NAKAI (APK200608) as an active ingredient and the pharmaceutically acceptable carriers or excipients, for the treatment and prevention of inflammatory, allergic or atopic dermatitis disease.

In accordance with another aspect of the present invention, there is also provided a use of novel purified extract from the extract of Agrimonia coreana NAKAI (APK200608) for manufacture of medicines employed for treating or preventing inflammatory, allergic or atopic dermatitis disease.

The term “pharmaceutically acceptable carriers or excipients” defined herein comprises pharmaceutical additives, the inactive ingredients used to make up a medication. They include dyes, flavors, binders, emollients, fillers, lubricants, preservatives, and many more classifications. Common excipients include cornstarch, lactose, talc, magnesium stearate, sucrose, gelatin, calcium stearate, silicon dioxide, shellac and glaze, which has been well-known in the art (See, Home-page of Food and Drug Administration: www.fda.gov or drug information online: www.drugs.com) or previous literature (for example, Rowe, Raymond C et al., Handbook of Pharmaceutical Excipients, Pharmaceutical Press, 7th Edition, 2012)

In accordance with another aspect of the present invention, there is also provided a method of treating or preventing inflammatory, allergic or atopic dermatitis disease in mammals, wherein the method comprises administering a therapeutically effective amount of purified extract from the extract of Agrimonia coreana NAKAI (APK200608) into the mammal suffering from inflammatory, allergic or atopic dermatitis diseases.

The inventive composition for treating and preventing inflammatory, allergic or atopic dermatitis disease may comprises above extracts as 0.1˜99%, preferably, 0.1˜50% by weight based on the total weight of the composition.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract varies depending on the condition and the weight of the subject, severity, drug form, route and period of administration, and may be chosen by those skilled in the art. However, in order to obtain desirable effects, it is generally recommended to administer at the amount ranging from 0.0001 to 1000 mg/kg, preferably, 0.001 to 100 mg/kg by weight/day of the inventive extract of the present invention. The dose may be administered in single or divided into several times per day.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

The inventive extract of the present invention also can be used as a main component or additive and aiding agent in the preparation of various functional health food and health care food.

Accordingly, it is the other object of the present invention to provide a health functional food comprising purified extract from the extract of Agrimonia coreana NAKAI (APK200608) for the prevention or alleviation of inflammatory, allergic or atopic dermatitis disease.

The term “a functional health food” defined herein the functional food having enhanced functionality such as physical functionality or physiological functionality by adding the extract of the present invention to conventional food to prevent or improve the purposed diseases in human or mammal.

It is the other object of the present invention to provide a health care food comprising purified extract from the extract of Agrimonia coreana NAKAI (APK200608) together with a sitologically acceptable additive for the prevention or alleviation of inflammatory, allergic or atopic dermatitis disease.

The term “a health care food” defined herein the food containing the extract of the present invention showing no specific intended effect but general intended effect in a small amount of quantity as a form of additive or in a whole amount of quantity as a form of powder, granule, capsule, pill, tablet etc.

The term “a sitologically acceptable additive”defined herein comprises “any substance the intended use which results or may reasonably be expected to result-directly or indirectly-in its becoming a component or otherwise affecting the charac-teristics of any food”, and can be classified into three groups according to its origin, i.e.,
(1) chemically synthetic additive such as ketones, glycine, potassium citrate,

nicotinic acid, etc;
(2) natural additive such as persimmon dye, licorice extract, crystalline cellulose, gua dum etc;
(3) the mixed additive therewith such as sodium L-glutamate, preservatives, tar dye

etc, or various categories according to its function in the food, for example, thickening agent, maturing agent, bleaching agent, sequestrant, humectant, anti-caking agent, clarifying agents, curing agent, emulsifier, stabilizer, thickener, bases and acid, foaming agents, nutrients, coloring agent, flavoring agent, sweetener, preservative agent, anti-oxidant, etc, which has been well-known in the art or previous literature (See, “Codex General Standard for Food Additives” (GSFA, Codex STAN 192-1995) in Home-page of GSFA Online: www.codexalimentarius.net/gsfaonline/index.html).

If a substance is added to a food for a specific purpose in that food, it is referred to as a direct additive and indirect food additives are those that become part of the food in trace amounts due to its packaging, storage or other handling.

The term “health care foods or health functional foods” disclosed herein can be contained in food, health beverage, dietary supplement etc, and may be formulated into a form of pharmaceutically dosing form such as a powder, granule, tablet, suspension, emulsion, syrup, chewing tablet, capsule, beverage etc; or the food form, for example, bread, rice cake, dry fruit, candy, chocolate, chewing gum, ice cream, milk such as low-fat milk, lactose-hydrolyzed milk, goat-milk, processed milk, milk product such as fermented milk, butter, concentrated milk, milk cream, butter oil, natural cheese, processed cheese, dry milk, milk serum etc, processed meat product such as hamburger, ham, sausage, bacon etc, processed egg product, fish meat product such as fish cake etc, noodle products such as instant noodles, dried noodles, wet noodles, fried noodles, non-fried noodles, gelatinized dry noodles, cooked noodles, frozen noodles, Pasta etc, tea product such as tea bag, leached tea etc, health drinks such as fruit drinks, vegetable drinks, carbonated soft drinks, soymilk drinks, lactic beverage mixed beverage, etc, seasoning food such as soy sauce, soybean paste, red pepper paste, chunjang (a kind of fermented soybean product colored by caramel), cheonggukjang (natural fermented soybean by B. subtillis), mixed paste, vinegar, sauce, ketchup, curry, dressing etc, margarine, shortening, pizza etc, but not intended herein to limit thereto, for preventing or improving of purposed disease.

Also, above described extract can be added to food or beverage for prevention and improvement of purposed disorder. The amount of above described extract in food or beverage as a functional health food or health care food may generally range from about 0.01 to 100 w/w % of total weight of food for functional health food composition. In particular, although the preferable amount of the extract of the present invention in the functional health food, health care food or special nutrient food may be varied in accordance to the intended purpose of each food, it is preferably used in general to use as an additive in the amount of the extract of the present invention ranging from about 0.01 to 5% in food such as noodles and the like, from 40 to 100% in health care food on the ratio of 100% of the food composition.

Providing that the health beverage composition of present invention contains above described extract or compound as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartame et al., may be useful favorably. The amount of above described natural carbohydrate is generally ranges from about 1 to 20 g, preferably 5 to 12 g in the ratio of 100 [symbol] of present beverage composition.

The other components than aforementioned composition are various nutrients, a vitamin, a mineral or an electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract or compound therein are various food, beverage, gum, vitamin complex, health improving food and the like.

Inventive extract of the present invention has no toxicity and adverse effect therefore; they can be used with safe.

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

The above and other objects, features and other advantages of the present invention will more clearly understood from the following detailed description taken in conjunction with the accompanying drawings, in which;

FIG. 1 shows HPLC analysis on apigenin-7-glucuronide (AP) and luteolin-7-glucuronide (LG) in comparative APH₂O/AP30%/AP70% extract isolated from Agrimonia pilosa prepared in comparative Example;

FIG. 2 shows HPLC analysis on rutin (RT), luteolin(LT), apigenin(AG), kaempferol (KP) and quercetin (QR) in comparative APH₂O/AP30%/AP70% extract isolated from Agrimonia pilosa prepared in comparative Example;

FIG. 3 shows standard curves of each components in inventive purified APK200608 extract from Agrimonia coreana NAKAI prepared in Example 2;

FIG. 4 shows HPLC analysis on rutin (RT), luteolin(LT), apigenin(AG), kaempferol (KP) and quercetin (QR) in inventive purified APK200608 extract from Agrimonia coreana NAKAI prepared in Example;

FIG. 5 shows HPLC analysis on alphitolic acid in APH₂O/AP30%/AP70% extract isolated from Agrimonia pilosa prepared in comparative Example;

FIG. 6 shows HPLC analysis on alphitolic acid in inventive purified APK200608 extract isolated from Agrimonia pilosa prepared in comparative Example;

FIG. 7 shows the inhibitory effect of the inventive purified extract/comparative APH₂O/AP30%/AP70% extract on the proliferation of CD4+ T cell;

FIG. 8 shows the inhibitory effect of the luteolin and alphitolic acid isolated therefrom on the proliferation of T cell;

FIG. 9 represents the inhibitory effect of the inventive purified extract/comparative APH₂O/AP30%/AP70% extract on the release of cytokines;

FIG. 10 represents the inhibitory effect of the inventive purified extract/comparative APH₂O/AP30%/AP70% extract on the degranulation of mast cell;

FIG. 11 presents the inhibitory effect of the inventive purified extract/comparative APH₂O/AP30%/AP70% extract on ORAI1.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Comparative Example, Examples and Experimental Examples are intended to further illustrate the present invention without limiting its scope.

### Comparative Example 1. The Preparation of Comparative Extract (1)

200 g of dried Agrimonia pilosa (Minkyunhmulsann Co. Ltd. Korea) was sliced to prepare following comparative examples.

### 1-1. 30% Ethanol Extract

20 fold volume of 30% ethanol(v/w) was added to 200 g of dried Agrimonia pilosa (Minkyunhmulsann Co. Ltd. Korea) and subjected to reflux extraction at 70° C. for 4 hours. After filtration of the extract through filter paper (pore size, 5 μm) to remove the debris, the filtrate was concentrated under vaccuo (−700-760 mmHg) at 40-50° C. to afford concentrated extract. The concentrated extract was dried with freeze drying process to obtain 20.8 g of comparative 30% ethanol extract (1) (designated as “AP30%” hereinafter)

### 1-1. 70% Ethanol Extract

20 fold volume of 70% ethanol(v/w) was added to 200 g of dried Agrimonia pilosa (Minkyunhmulsann Co. Ltd. Korea) and subjected to reflux extraction at 70° C. for 4 hours. After filtration of the extract through filter paper (pore size, 5 μm) to remove the debris, the filtrate was concentrated under vaccuo (−700-760 mmHg) at 40-50° C. to afford concentrated extract. The concentrated extract was dried with freeze drying process to obtain 21.2 g of comparative 70% ethanol extract (2) (designated as “AP70%” hereinafter)

### 1-2. Water Extract

20 fold volume of distilled water (v/w) was added to 200 g of dried Agrimonia pilosa (Minkyunhmulsann Co. Ltd. Korea) and subjected to reflux extraction at 90° C. for 4 hours. After filtration of the extract through filter paper (pore size, 5 μm) to remove the debris, the filtrate was concentrated under vaccuo (−700-760 mmHg) at 40-50° C. to afford concentrated extract. The concentrated extract was dried with freeze drying process to obtain 15.7 g of comparative water extract (2) (designated as “APH₂O” hereinafter)

### 1-3. Component Analysis

The component analysis on the active ingredient of AP30% and APH2O was performed using by HPLC (Agilent 1260 model, USA) according to the condition in Table 1.

The test result on component analysis of luteolin-7-glucuronide (LG) and apigenin-7-glucuronide (AG) was shown in FIG. 1 and that of rutin (RT), luteolin (LT), apigenin (AP), kaempferol l(KP) and quercetin (QR) was shown in FIG. 2.

TABLE 1
HPLC condition
HPLC condition

| Apparatus | HPLC (Agilent 1290 Series, Agilent) | | |
|---|---|---|---|
| condition | | | |
| Column | YMC-Triart C18 (100 * 2.1 mm, 5 μm) | | |
| Column tem- | 25° C. | | |
| perature | | | |
| Mobile phase | A: 0.05% formic acid | | |
| | B: 0.05% formic acid/Acetonitrile | | |
| UV Absorbance | rutin | | 266 nm |
| | Quercetin | | |
| | Kaempferol | | 265 nm |
| | Luteolin | | 340 nm |
| | Apigenin | | |
| | Luteolin-glucuronide | | |
| | Apigenin-7-glucuronide | | |
| Mobile phase | time | Mobile phase A (%) | Mobile phase B (%) |
| | 0 | 95 | 5 |
| | 3 | 90 | 10 |
| | 5 | 70 | 30 |
| | 10 | 60 | 40 |
| | 12.5 | 10 | 90 |
| | 15 | 10 | 90 |

| | |
|---|---|
| Injection volume | 10 μl |
| Flow rate | 0.3 ml/min |

As can be seen in FIG. 1, it has been confirmed that each ingredient in APH2O extract was detected at 6.393 mins (luteolin-7-glucuronide) and 6.821 mins (apigenin-7-glucuronide), and that in AP30% was detected at 6.399 mins (luteolin-7-glucuronide) and 6.835 min (apigenin-7-glucuronide) respectively.

As can be seen in FIG. 2, it has been confirmed that each ingredient in APH2O extract was detected at 8.928 mins (luteolin), 10.608 mins (apigenin), 9.051 mins (quercetin). However, kaempferol was not detected and each ingredient in AP30% was detected at 8.953 mins (luteolin), 10.636 mins (apigenin), 11.029 mins (kaempferol), 9.075 mins (quercetin). respectively.

The standard curve of each ingredients was shown in FIG. 3 for analysis of each content (μg/mg) based on HPLC pattern (retention time) shown in FIGS. 1 and 2.

The content of each ingredient (%) in the sample was calculated based on the HPLC pattern (retention time) and HPLC test result was shown in Table 2.

TABLE 2
HPLC result (APW and AP30%)

| | | Comparative Example 1 | |
|---|---|---|---|
| Active ingredient | extract | Retention Time (mins) | Content (μg/mg) |
| Luteolin-7- | APH₂O | 6.393 | 5.110 |
| glucuronide | AP30% | 6.399 | 6.872 |
| Apigenin-7- | APH₂O | 6.821 | 6.691 |
| glucuronide | AP30% | 6.404 | 9.428 |
| Luteolin | APH₂O | 8.928 | 0.021 |
| | AP30% | 8.953 | 0.046 |
| Apigenin | APH₂O | 10.608 | 0.010 |
| | AP30% | 10.636 | 0.028 |
| Kaempferol | APH₂O | ND (no detected) | ND |
| | AP30% | 11.029 | 0.009 |
| quercetin | APH₂O | 9.051 | 0.042 |
| | AP30% | 9.075 | 0.058 |

### Example 1. Identification of Origin of Test Sample Through Gene Analysis

In order to identify the origin of test sample procured from conventional company (Minkyunhmulsann Co. Ltd. Korea, 143, dong-ro, Jeongneungcheondong-ro, Dongdaemun-gu, Seoul, Republic of Korea), Korean gene analysis company (Geno Tech Corp., www.genotech.co.kr/26-69, Gajeongbuk-ro, Yuseong-gu, Daejeon, Republic of Korea,T;82-42-862-8404) has performed gene analysis to determine whether the leaf of test sample is Agrimonia coreana NAKAI or not according to following procedure.

### 1-1. Test Procedure

### 1-1-1. DNA Extraction and Purification

The DNA extraction and purification from test sample was performed according to CTAB method (Schlaeger TM et al., Proc. Natl. Acad. Sci. USA, 94(7), pp.3058-3063, 1997 cetyl trimethylammonium bromide method, Adam Healey, Agnelo Furtado, Tal Cooper & Robert J Henry. Protocol: a simple method for extracting next-generation sequencing quality genomic DNA from recalcitrant plant species. Plant Methods 2014, 10:21) using by conventional reagents provided from company according to instruction manual of the company.

### (1) Pre-Treatment of Test Sample

100 g of dried test sample was poured to mortar and pulverized to be very fine powder using liquid nitrogen for DNA extraction.

### (2) DNA Extraction Using by CTAB Method

The dried powder was transferred to 2 ml of tube and 500 μL of lysis buffer (K-3021, BIONEER Corp.,Korea), 20 mM Tris-HCl (pH 8.0), 2 mM sodium EEDTA, 1.2% Triton X-100, 20 mg/mL of lysozyme and 10 μL of proteinase K solution (K-3031, BIONEER Corp.,Korea, more than 600 mAU/mL) were added thereto to mix thoroughly and reacted together for 1 hour at 37° C. in pyrostat (BS-31, Vison Lab Science Co. Ltd, Korea).

400 μL of CTAB (cetyl trimethylammonium bromide) buffer solution (C2007, Biosesang Co. Ltd. Korea) containing 0.1M Tris (pH 8.0), 1.4M NaCl, 0.02M EDTA (pH8.0), 2% hexadecyltrimethylammonium bromide and 0.2% 2-mercaptoethanol, was added thereto and reacted together for 30 mins at 65° C. in pyrostat (BS-31, Vison Lab Science Co. Ltd, Korea).

600 μL of solvent mixture (Phenol:Chloroform:Isoamylalcohol=25:24:1) and 300 μL of distilled water were added to the reactant, mixed thoroughly and centrifuged for mins at 20° C. at the speed of 14,000 rpm.

600 μL of supernatant was added to new 1.5 mL tube and 600 μL of isopropanol was added thereto to mix together, react for 10 mins at room temperature and centrifuged for 10 mins at 20° C. at the speed of 14,000 rpm.

After removing the remaining supernatant, the precipitated DNA was washed with 500 μL of 70% ethanol three times and left alone to dry purified DNA at room temperature.

The purified DNA was dissolved in 20-30 μL of sterilized triple distilled water to left alone for 1 hour at 4° C. and 2 μL of RNase enzyme (100 mg/mL, 7,000 units/mL, K-3031, BIONEER Corp., Korea) was added thereto to react together for 30 mins at 37° C.,

### (3) Determination of DNA Purity and DNA Content

After confirming the extracted DNA through electrophoresis using by 1% agarose gel (54801, Takara Korea Biomedical Inc.), DNA was stained with staining agent (EtBr: Ethidium Bromide, E1510, Sigma-Aldrich Co. Ltd.) and the purity and content of DNA were determined using by UV spectrophotometer (Nanodrop, USA) under UV light (Absorbance: 260 nm and 280 nm, Gel Doc XR, Bio-RAD) to DNA quantification.

### 1-1-2. PCR Condition for DNA Barcode Analysis

### (1) DNA Barcode Primer

The DNA barcode primers shown in Table 3 was used in the experiment and the specific DNA barcode for Agrimonia coreana NAKAI was developed by comparing with barcode information listed in GenBank DB.

TABLE 3
DNA bar-code primers used in DNA analysis
*DNA bar-code primers used in DNA analysis

| Target region | | | Primer | Primer Sequence | Sequence I.D. |
|---|---|---|---|---|---|
| cpDNA | Coding | matK* | 3F_KIM f | CGTACAGTACTT | 1 |
| | region | | | TTGTGTTTACGA | |
| | | | | G | |
| | | | 1R_KIM r | CCCATTCATCTG | 2 |
| | | | | GAAATCTTGGTT | |
| | | | | C | |
| | | rbcL* | rbcLa_R | GTAAAATCAAGT | 3 |
| | | | | CCACCGCG | |
| | | | rbcLa_F | ATGTCACCACAA | 4 |
| | | | | ACAGAGACTAA | |
| | | | | AGC | |
| | Non- | atpF-atpH* | atpF f | TCGCTTAACACT | 5 |
| | coding | | | CCCCTTCC | |
| | region | | atpH r | GCTTTCATGGAA | 6 |
| | | | | GCTTTAACAAT | |
| | | psbA-trnH* | psbA3 F | GTTATGCATGAA | 7 |
| | | | | CGTAATGCTC | |
| | | | trnHf_05 | CGCGCGTGGTGG | 8 |
| | | | | ATTCACAATCC | |
| | | psbK-psbI* | psbK f | TTAGCCTTTGTT | 9 |
| | | | | TGGCAAG | |
| | | | psbI r | ATAGTTTAAGAG | 10 |
| | | | | TAAGCAT | |
| | | trnL-intron** | trn c | CGAAATCGGTAG | 11 |
| | | | | ACGCTACG | |
| | | | trn d | GGGGATAGAGG | 12 |
| | | | | GACTTGAAC | |
| | | trnL-trnF** | trn e | GGTTCAAGTCCC | 13 |
| | | | | TCTATCCC | |
| | | | trn f | ATTTGAACTGGT | 14 |
| | | | | GACACGAG | |

*, CBOL; A CBOL Plant working group(2009) A DNA barcode for land plants. Proc Nati Acad Sci USA. 106(31): 12794-12797.
**, trnT-trnL; trnL-intron, trnL-trnF; Taberlet et al.(1991) Universal ※primers for amplification of three non-coding regions of chloroplast DNA. Plant Molecular Biology 17: 1105-1109.
※ underlined character: specific base sequences of Agrimonia genus

### (1) PCR (Polymerase Chain Reaction)

For PCR condition using by the barcode primers, the mixture of 2 μL of forward primer (10 pmole/μL), 2 μL of reverse primer (10 pmole/μL) and 20 ng/μL of fixed quantity of stranded DNA was mixed with PCR premix (K-2115, Bioneer Corp., Korea) and added with distilled water to the extent that the final reaction volume has reached to be 30 μL.

The isolated DNA was amplified by PCR [(pre-denaturation at 95° C. for 5 min, de-naturation at 95° C. for 45 sec, annealing at 55° C. for 45 sec and extension at 72° C. for 1 min.)X35 cycles and final-extension at 72° C. for 5 mins] to afford the amplified DNA product.

5 μL of the amplified DNA product was dropped into 1% agarose gel to be electrophoresed and treated with EtBr (Ethidium Bromide) staining method to confirm the presence of amplified products.

### (2) DNA Barcode Sequencing Analysis

The PCR product prepared from the above-step was purified to be used for performing following DNA barcode sequencing analysis.

The analyzed DNA sequence of each sample by each DNA barcode was aligned by multiple alignment method according to Bioedit program (version 7.0.5.3 Tom Hall Ibis Biosciences, USA) as well as Clustal method (D Thompson, D G Higgins, T J Gibson. CLUSTAL W: improving the sensitivity of progressive multiple sequence alignment through sequence weighting, position-specific gap penalties and weight matrix choice. Nucleic Acids Res. 1994 Nov. 11;22(22):4673-80).

The difference between each species of plant has been confirmed by analyzing the positions of insertion, deletion and substitution of each DNA based on the sequencing result.

### (3) Blast Analysis: NCBI GenBank DB

The analyzed DNA sequence of each sample by each DNA barcode was analyzed by comparing with 7 barcode region of Chloroplast DNA (cpDNA) of sample. i.e. matK, rbcL, atpF-atpH, psbA-trnH, psbK-psbI, trnL-intron, trnL-trnF.

It has been confirmed that 7 barcode region of Chloroplast DNA (cpDNA) of sample (matK, rbcL, atpF-atpH, psbA-trnH, psbK-psbI, trnL-intron, trnL-trnF) are same as those of Agrimonia coreana NAKAI, of which result confirmed that the leaf of test sample is identified as Agrimonia coreana NAKAI.

### Example 2. The Preparation of Inventive Novel Purified Extract (1)

### 2-1. Preparation of Crude Extract (APE)

1 kg of dried leaf of Agrimonia coreana NAKAI of which species has been identified in Example 1 was cut into small pieces and mixed with 20 fold volume of 70% ethanol(v/w). The mixture was extracted with reflux extraction at 70° C. for 4 hours to collect the filtrate, three times. The extract was filtered with filtration apparayus (R5PPMF01ES, Kajika Co.,) to remove the debris, five times. The collected filtrate was concentrated by rotary vacuum evaporator (Buchi, Ratavapor, R-300) at 55˜65° C. under reduced pressure and dried with freezing dryer to obtain 460 g of dried crude extract (designated as “APE” hereinafter).

### 2-2. Preparation of Inventive Purified Extract (APK200608)

The crude extract (APE) preppared in Example 2-1, was loaded on open column chromatography using by highly porous styrene non-polar adsorbent (Diaion HP20) as a stationary phase with eluting the suspension using by eluting solvent (distilled water: ethanol=70:30→25:75) to afford the 1^{st} eluted purified extract. The 1^{st} eluted purified extract was then loaded on open column chromatography using by highly porous styrene non-polar adsorbent (Diaion HP20) as a stationary phase with eluting the suspension using by eluting solvent (distilled water: ethanol=50:50→5:95) to afford the 2^{nd} eluted purified extract. The purification steps was repeated five times to collect the 2^{nd} eluted purified extract and concentrated under reduced pressure to afford dried 150 g of the inventive purified extract (APK200608) showing potent anti-inflammatory activity, of which preparation provides industrially useful mass-production.

### 2-3. HPLC Analysis of Inventive Purified Extract (APK200608)

The component analysis on inventive purified extract (APK200608) was performed using by HPLC (Agilent 1260 model, USA) according to the condition in Table 1 and the result was shown in FIG. 4.

As can be seen in FIG. 4, it has been confirmed that each ingredient in APK200608 extract was detected at 9.080 mins (quercetin, 254 nm), 11.046 mins (kaempferol, 265 nm), 6.400 mins (luteolin-7-glucuronide, 340 nm), 6.831 mins (apigenin-7-glucuronide, 340 nm), 8.954 mins (luteolin, 340 nm) and 10.643 mins (apigenin, 340 nm), respectively.

Each content (μg/mg) of active ingredients was calculated based on HPLC pattern (retention time) shown in FIG. 4 and the calculated result was shown in Table 4.

As can be seen in Table 4, it has been confirmed that APK200608 extract prepared by the inventive preparation method for mass-production, showed the higher amount of anti-inflammatory active ingredients such as luteolin-7-glucuronide, apigenin-7-glucuronide, (luteolin, apigenin, kaempferol, and, quercetin, especially, 4.86 fold amount of luteolin and 7.3 fold amount of apigenin, respectively, comparing with APH₂O/AP30% extract prepared by the conventionally known preparation method from Agrimonia pilosa used as a comparative examples.

TABLE 4
HPLC result (APK200608)

| Active | | Comparative Example 1 | |
|---|---|---|---|
| ingredient | Extract | Retention Time (mins) | Content (μg/mg) |
| Luteolin-7- | APK200608 | 6.400 | 10.704 |
| glucuronide | | | |
| Apigenin-7- | APK200608 | 6.831 | 13.546 |
| glucuronide | | | |
| Luteolin | APK200608 | 8.954 | 0.102 |
| Apigenin | APK200608 | 10.643 | 0.073 |
| Kaempferol | APK200608 | 11.046 | 0.021 |
| quercetin | APK200608 | 9.080 | 0.079 |

### Example 3. Newly Isolated Compound from Agrimonia coreana NAKAI

The new component analysis on inventive purified extract (APK200608) from Agrimonia coreana NAKAI as well as APE/AP30% extract from Agrimonia pilosa was performed using by HPLC (Agilent 1260 model, USA) according to the condition in Table 5 (result: FIG. 5) and Table 6 (result: FIG. 6) and the result was shown in FIGS. 5 and 6.

TABLE 5
HPLC condition for detection of alphitolic acid in APE/AP30% extract
HPLC condition

| Apparatus | HPLC (HITACHI Chromaster, HITACHI, Japan) | | |
|---|---|---|---|
| condition | | | |
| Column | TSK-gel 100V (4.6 mm × 250 mm, 5 μm), TOSOH, | | |
| | Japan | | |
| Column tem- | 25° C. | | |
| perature | | | |
| Mobile phase | A: acetonitrile | | |
| | B: 5% aqueous acetonitrile/0.04% TFA | | |
| Detection | 204 nm | | |
| wavelength | | | |
| (UV absorbance) | | | |
| Mobile phase | time | Mobile phase A (%) | Mobile phase B (%) |
| | 0 | 75 | 25 |
| | 16 | 75 | 25 |

| | |
|---|---|
| Injection volume | 10 μl |
| Flow rate | 1 ml/min |

TABLE 6
HPLC condition for detection of alphitolic acid in APK200608 extract
HPLC condition

| Apparatus | HPLC (HITACHI Chromaster, HITACHI, Japan) | | |
|---|---|---|---|
| condition | | | |
| Column | TSK-gel 100V (4.6 mm × 250 mm, 5 μm), TOSOH, | | |
| | Japan | | |
| Column tem- | 25° C. | | |
| perature | | | |
| Mobile phase | A: acetonitrile | | |
| | B: 5% aqueous acetonitrile/0.04% TFA | | |
| Detection | 205 nm | | |
| wavelength | | | |
| (UV absorbance) | | | |
| Mobile phase | time | Mobile phase A (%) | Mobile phase B (%) |
| | 0 | 70 | 30 |
| | 2 | 70 | 30 |
| | 20 | 100 | 0 |
| | 26 | 100 | 0 |

| | |
|---|---|
| Injection volume | 10 μl |
| Flow rate | 1 ml/min |

As can be seen in FIG. 5, it has been confirmed that APH20/AP30% extract from Agrimonia pilosa has not found to detect alphitolic acid.

In the contrary, it has been confirmed that inventive APK200608 extract from Agrimonia coreana NAKAI has found to contain newly isolated compound, i.e., alphitolic acid in an amount of 0.00152% (w/w) at 10 mins, as can be seen in Table 7 and FIG. 6.

TABLE 7
component analysis of alphitolic acid in APK200608 extract

| | Calibration | Final | Weight of | | |
|---|---|---|---|---|---|
| | curve result | volume | sample | Content | Content |
| Extract | ((μg/mL) | (mL) | (mg) | (%) | (mg/mL) |
| APK200608 | 1.9330 | 0.4 | 40.6 | 0.00152 | 0.0152 |

### Experimental Example 1. Inhibition Effect on T Cell Proliferation

In order to determine the anti-immunological activity of inventive extract, following inhibition test of proliferation of T cell isolated from human blood was performed according to the procedure disclosed in the literature (J Immunol Jul. 15, 2002, 169 (2) 802-808).

### 1-1. Isolation of T Cell from Human Blood

In order to evaluate the anti-immunological activity of inventive extract, PBMC(peripheral blood mononuclear cell) and T cell which had been used in the various immunological analysis, were prepared from human blood using Fiicoll (GE17-5442-02, GE Healthcare) according to the previous literature (BIOPRESERVATION AND BIOBANKING, Volume 14, Number 5, 2016).

CD4+ T cell was carefully isolated from the isolated PBMC using by T cell isolation kit (130-096-533, Miltenyi biotec.) according to the manufacture's manual instruction.

### 1-2. Evaluation of Inhibitory Effect on T Cell Proliferation Using by CFSE

The anti-immunological effect on T cell was evaluated by the inhibition effect on the proliferation of CD4+ T cell.

CFSE (Carboxyfluorescein diacetate succinimidyl ester, C34570, ThermoFisher Scientific) was treated to the isolated T cell to be 1 μM to incubate for 30 mins at room temperature in an anti-glare condition and washed with DPBS (Dulbecco's Phosphate-Buffered Saline, LB 001-01, welgene) twice to finish staining the cell.

The stained T cells with CFSE were inoculated into 96 well plates (2×10⁵ cells/well) and stimulated with anti-CD3 and anti-CD 28.

In order to determine the inhibition effect on the proliferation of T cell of inventive extract, the test samples, i.e., the extract disclosed in Comparative Examples and Examples, were inoculated into the well plate to be 100 μg/mL with the stimuli of anti-CD3 and anti-CD 28.

10 μM BTP2([N-{4-[3,5-bis(Trifluoromethyl)-1H-pyrazol-1-yl]phenyl}-4-methyl-1,2,3-thiadiazole-5-carboxamide], 203890-M, Calbiochem) was used as a control group.

The T cell treated with test sample was incubated at 37° C. for 72 hours in 5% CO2 incubator (MCO-18AC, Panasonic) and then the cell was collected to be analyzed using Flow cytometry (BD LSRFortessa, BD Biosciences).

As can be seen in FIGS. 7 and 8, it has been confirmed that there has no proliferation of T cell in the group treated without anti-CD3 and anti-CD 28 ((−)CD3, CD28) whereas there has sufficient proliferation of T cell in the group treated with anti-CD3 and anti-CD 28 ((+)CD3, CD28).

Additionally, it has been confirmed that there has sufficient proliferation of T cell in the group treated with BTP2, similarly to the result in the group treated without anti-CD3 and anti-CD 28 ((−)CD3, CD28).

Finally, it has been confirmed that the proliferation of T cell in the group treated with the inventive APK200608 isolated from Agrimonia coreana NAKAI has been unexpectedly inhibited (89.00±1.191%) comparing with the group treated with APH2O (36.04±2.335%) or AP30% (39.67±1.889%) isolated from Agrimonia pilosa used as comparative Examples (See Table 8).

TABLE 8
inhibition effect on CD4+ T cell proliferation

| Test sample | Inhibition rate on T cell proliferation (%) |
|---|---|
| BTP2 | 98.84 ± 0.0006 |
| APH₂0 | 36.04 ± 2.335 |
| AP30% | 39.67 ± 1.889 |
| APK200608 | 89.00 ± 1.191 |

### Experimental Example 2. Inhibition Effect on IL-2 Release in Jurkat T Cell

In order to determine the anti-immunological activity of inventive extract, following inhibition test of cytokine release in Jukrat T cell was performed according to the procedure disclosed in the literature (ARTHRITIS & RHEUMATISM Vol. 52, No. 9, September 2005, pp 2730-2739).

ELISA (Enzyme-linked immunosorbent assay) was performed to determine the release of IL-2 in Jukrat T cell.

Jukrat T cell (TIB-152, American Type Culture Collection(ATCC)) was inoculated into 96 well plates (2×10⁵ cells/well) and stimulated with anti-CD3 and anti-CD 28.

In order to determine the inhibition effect on the IL-2 release of T cell of inventive extract, the test samples, i.e., the extract disclosed in Comparative Examples and Examples, were inoculated into the well plate to be 100 μg/mL with the stimuli of anti-CD3 and anti-CD 28.

10 μM BTP2([N-{4-[3,5-bis(Trifluoromethyl)-1H-pyrazol-1-yl]phenyl}-4-methyl-1,2,3-thiadiazole-5-carboxamide], 203890-M, Calbiochem) was used as a control group.

The T cell treated with samples was incubated at 37° C. for 72 hours in 5% CO2 incubator (MCO-18AC, Panasonic) and then Jukrat T cell was removed from the cultured fluid using by centrifuge (combi 514R, Hanil chem. Co. Korea) to collect only the cultured fluid.

The release of cytokines was analyzed by IL-2 specifically reacting ELISA kit (BGK60568, Peprotech Inc. NJ, USA) and the content of IL-2 was determined according to the instruction manual of manufacture.

As can be seen in FIG. 9, it has been confirmed that the increased release of IL-2 with the stimuli of (+)CD3 and CD28 in the group treated with BTP2, was reduced (90.30±0.552%),

Finally, it has been confirmed that the increased release of IL-2 with the stimuli of (+)CD3 and CD28 in the group treated with the inventive APK200608 isolated from Agrimonia coreana NAKAI has been unexpectedly reduced (88.46±1.209%) comparing with the group treated with APH2O (5.48±4.911%) or AP30% (30.29±4.613%) isolated from Agrimonia pilosa used as comparative Examples (See Table 9).

TABLE 9
inhibition effect on IL-2 release in T cell

| Test sample | Inhibition rate on IL-2 release (%) |
|---|---|
| BTP2 | 90.30 ± 0.552 |
| APH₂O | 5.48 ± 4.911 |
| AP30% | 30.29 ± 4.613 |
| APK200608 | 88.46 ± 1.209 |

### Experimental Example 3. Inhibition Effect on Degranulation in Mast Cell

In order to determine the anti-allergic activity of inventive extract, following inhibition test of degranulation in mast cell (RBL-2H3) using by beta-hexosaminidase assay was performed according to the procedure disclosed in the literature (INTERNATIONAL JOURNAL OF MOLECULAR MEDICINE 33: 469-477, 2014).

After treating IgE (D8406, Sigma) recognizing DNP(Dinitrophenyl) as an antigen to RBL-2H3 cell (CRL-2256, ATCC), the cell was incubated for 3 hours in the incubator (MCO-18AC, Panasonic) and then washed twice.

Both of 100 μg/mL of the test samples 1 μg/mL of DNP(Dinitrophenyl, A6661, Sigma) were inoculated into the cell and incubate for 1 hour in 5% CO2 incubator (MCO-18AC, Panasonic) to induce their degranulation.

The degranulation-induced cell cultured medium was reacted with 1 mM p-nitrophenyl N-acetyl-beta-D-glucosamine (N9376, Sigma) in buffer solution containing 50 mM Citric acid (C1909, Sigma) and 50 mM Tri-Na+ citrate dyhydrate (S1804, Sigma) for 1 hour and the reaction was finished with the buffer solution containing 50 mM Na⁺ carbonate deca hydrate (71360, Sigma) and 50 mM Na⁺ bicarbonate (S5761, Sigma) to determine the absorbance at 405 nm by microplate reader (Epoch, Biotek).

As can be seen in FIG. 10, it has been confirmed that the release of beta-hexosaminidase induced by the stimuli of DNP in DNP-IgE bound RBL-2H3 cell in the positive group treated with DNP((+) DNP), was more increased comparing with the negative control group treated with no DNP((−) DNP).

Finally, it has been confirmed that the release of beta-hexosaminidase induced by the stimuli of DNP in DNP-IgE bound RBL-2H3 cell in the group treated with the inventive APK200608 isolated from Agrimonia coreana NAKAI has been unexpectedly inhibited (75.15±4.56%) comparing with the group treated with APH2O (no inhibition) or AP30% (24.14±2.82%) isolated from Agrimonia pilosa used as comparative Examples (See Table 10).

TABLE 10
inhibition effect on the release of beta-hexosaminidase

| | Inhibition rate on the release of beta- |
|---|---|
| Test sample | hexosaminidase (%) |
| APH₂O | 0 |
| AP30% | 24.14 ± 2.82 |
| APK200608 | 75.15 ± 4.56 |

### Experimental Example 4. Inhibition Effect on ORAI1 Ion Channel Regulating Calcium Signaling in Cell

In order to determine the anti-immunolgical activity of inventive extract, following inhibition test of ORAI1 ion channel was performed according to the procedure disclosed in the literature (The American Journal of Chinese Medicine, Vol. 47, No. 7, 1-15).

It has been reported that the activation of an immune cell such as the proliferation of T cell, the reproduction and release of cytokines etc, is induced by the increased calcium level in cells through ORAI1 ion channel and an increase in intracellular levels of calcium ions (Ca2+) results from the engagement of immunoreceptors, such as the T-cell receptor, B-cell receptor and Fc receptors, as well as chemokine and costimulatory receptors. T (Nat Rev Immunol. 2007 Sep;7(9):690-702.).

The inhibition effect on ORAI1 ion channel which can increase the cellular calcium signaling pathway, an important pathway to induce the proliferation of T cell.

The extracellular fluid for patch clamp analysis was prepared to contain 135 mM NaCl, 3.6 mM KCl, 1 mM MgCl2, 10 mM CaCl2, 5 mM D-glucose, 10 mM HEPES(H3375, Sigma) and adjusted to pH 7.4 with NaOH. The intracellular fluid for patch clamp analysis was prepared to contain 130 mM Cs-glutamate, 20 mM BAPTA, 1 mM MgCl2, 3 mM MgATP, 0.002 mM sodium pyruvate, 20 mM HEPES (H3375, Sigma) and adjusted to pH 7.2 with CsOH (C8518, Sigma).

The ORAI1 electric current was determined by using Axopatch 200B amplifier (Axon Axopatch 200B Microelectrode Amplifier, Molecular Devices) and Digidata 1440A(Axon digidata 1440A, Molecular Devices) and the data was analyzed by pClamp10.4(pClamp10.4, Molecular Devices).

The ramp-pulse for recording electric current was changed to −130 mV˜50 mV for 100 msec and the voltage of cellular membrane was fixed to −10 mM to record every 20 seconds, repeatedly.

In order to compare the inhibition degree of test samples showing anti-immunological activity, 10 μM BTP2 (2038890-M, Calbiochm), a ORAI1 inhibitor, was poured into the chamber to confirm the inhibition of electric current at the end of determination.

As can be seen in FIG. 11, it has been confirmed that the activated ORAI1 electric current in the group treated with the inventive APK200608 isolated from Agrimonia coreana NAKAI has been unexpectedly inhibited (74.14±2.428%) comparing with the group treated with APH2O (21.83±2.832%)) or AP30% (56.16±1.232%) isolated from Agrimonia pilosa used as comparative Examples (See Table 11).

TABLE 11
inhibition effect on activated ORAI1 electric current

| | Inhibition rate on activated ORAI1 |
|---|---|
| Test sample | electric current (%) |
| APH₂O | 21.83 ± 2.832 |
| AP30% | 56.16 ± 1.232 |
| APK200608 | 74.14 ± 2.428 |

### Advantageous Effects of Invention

As described in the present invention, the present inventors demonstrated that the novel industrialized method for preparing purified extract (APK200608) from Agrimonia coreana NAKAI, a Korean native plant, can provide more abundant active ingredients showing potent ant-inflammatory activity comparing with the crude extract prepared by the conventional method disclosed in the prior art or previous known extract of Agrimonia pilosa; as well as the inventive purified extract showed more potent anti-inflammatory, anti-allergy and anti-atopic activity than that prepared by the conventional preparation method through various tests such as inhibition effect on T cell proliferation (Experimental Example 1), inhibition effect on IL-2 release in Jurkat T cell (Experimental Example 2), inhibition effect on degranulation in mast cell (Experimental Example 3), inhibition effect on ORAI1 ion channel regulating calcium signaling in cell (Experimental Example 4), therefore, it is confirmed that inventive purified extract is very useful in the alleviation or treatment of inflammatory disease, allergic disease and atopic dermatitis disease.

### BRIEF DESCRIPTION OF DRAWINGS

The above and other objects, features and other advantages of the present invention will more clearly understood from the following detailed description taken in conjunction with the accompanying drawings, in which;

FIG. 1 shows HPLC analysis on apigenin-7-glucuronide (AP) and luteolin-7-glucuronide (LG) in comparative APH₂O/AP30%/ AP70% extract isolated from Agrimonia pilosa prepared in comparative Example;

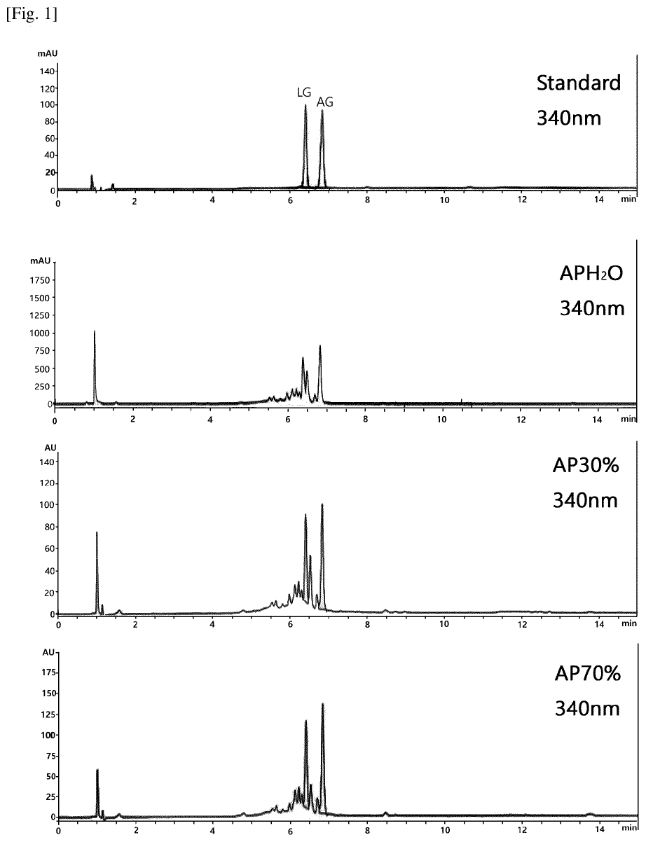

FIG. 2 shows HPLC analysis on rutin (RT), luteolin(LT), apigenin(AG), kaempferol (KP) and quercetin (QR) in comparative APH₂O/AP30%/AP70% extract isolated from Agrimonia pilosa prepared in comparative Example;

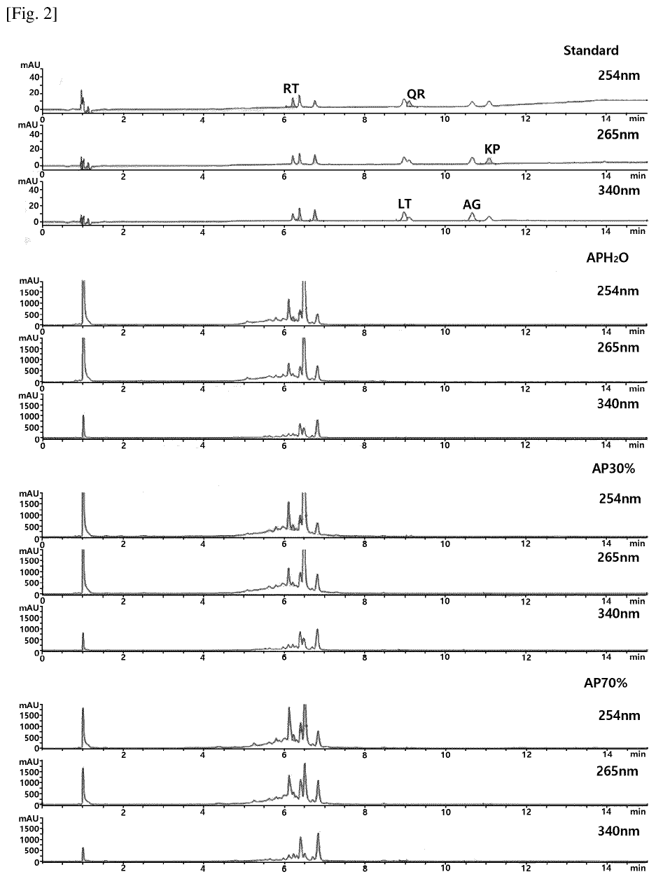

FIG. 3 shows standard curves of each components in inventive purified APK200608 extract from Agrimonia coreana NAKAI prepared in Example 2;

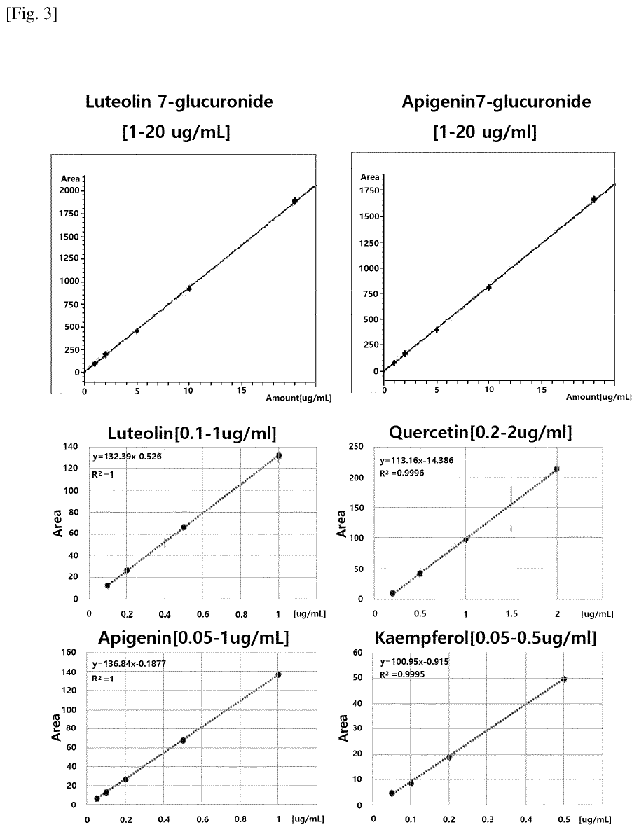

FIG. 4 shows HPLC analysis on rutin (RT), luteolin(LT), apigenin(AG), kaempferol (KP) and quercetin (QR) in inventive purified APK200608 extract from Agrimonia coreana NAKAI prepared in Example;

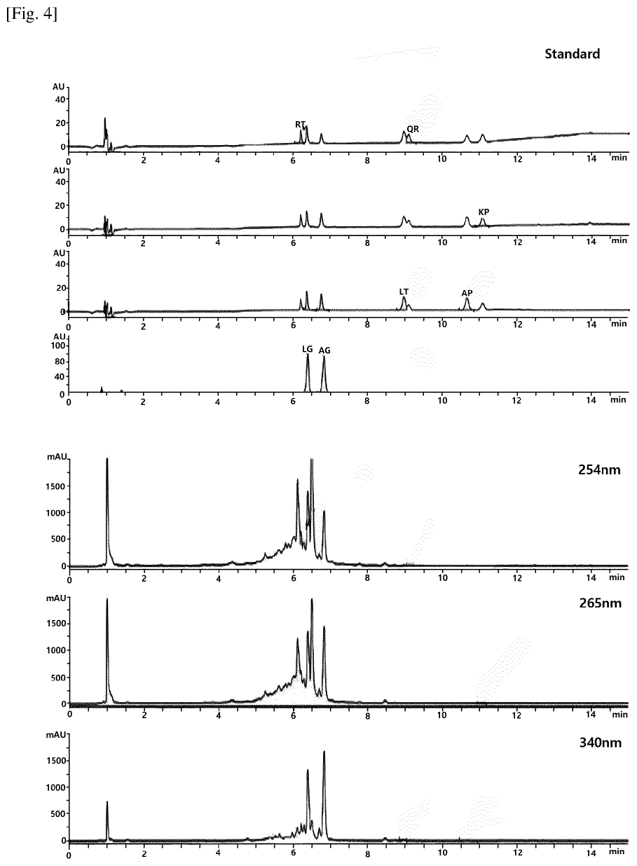

FIG. 5 shows HPLC analysis on alphitolic acid in APH₂O/AP30%/AP70% extract isolated from Agrimonia pilosa prepared in comparative Example;

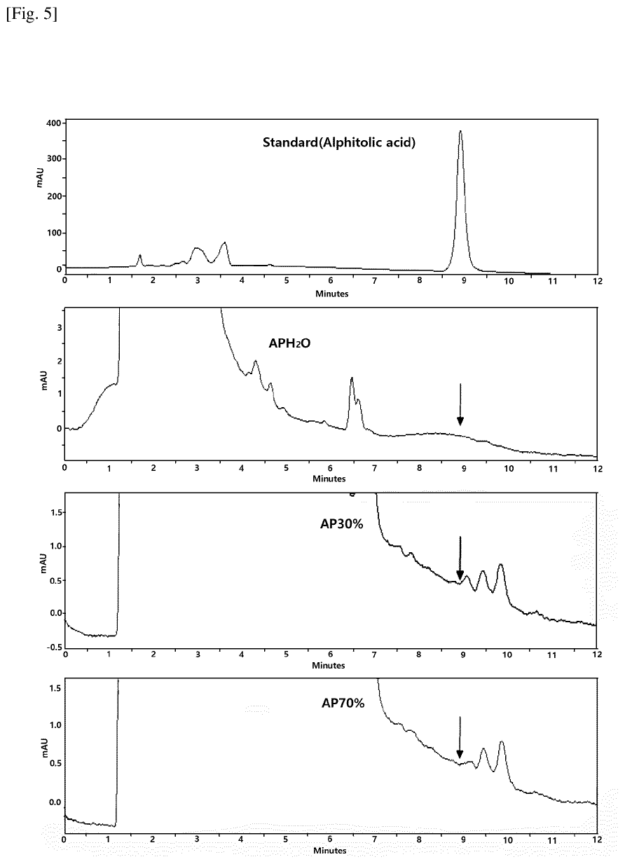

FIG. 6 shows HPLC analysis on alphitolic acid in inventive purified APK200608 extract isolated from Agrimonia pilosa prepared in comparative Example;

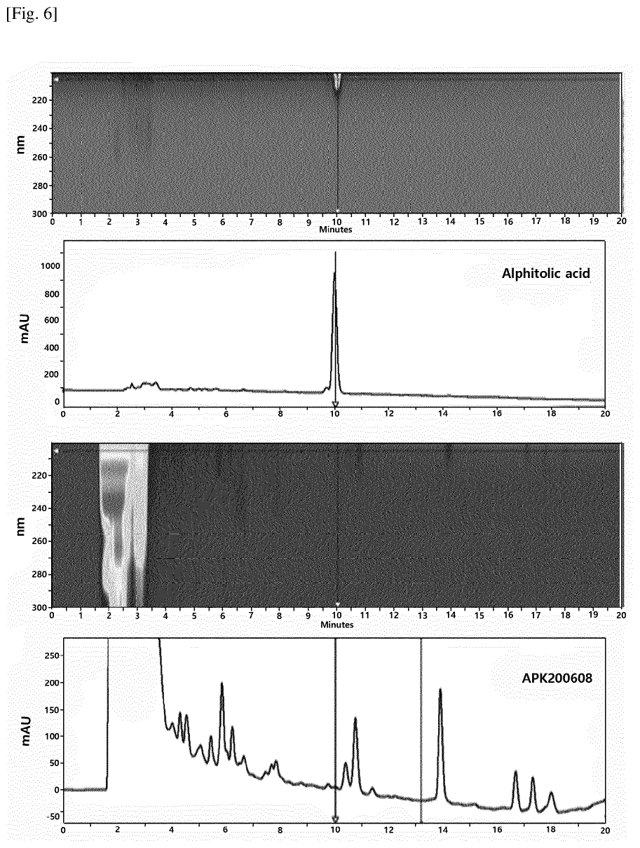

FIG. 7 shows the inhibitory effect of the inventive purified extract/comparative APH₂O/AP30%/ AP70% extract on the proliferation of CD4+ T cell;

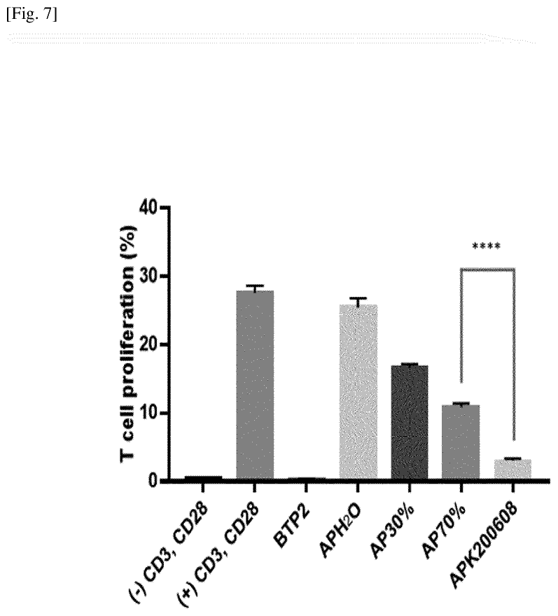

FIG. 8 shows the inhibitory effect of the luteolin and alphitolic acid isolated therefrom on the proliferation of T cell;

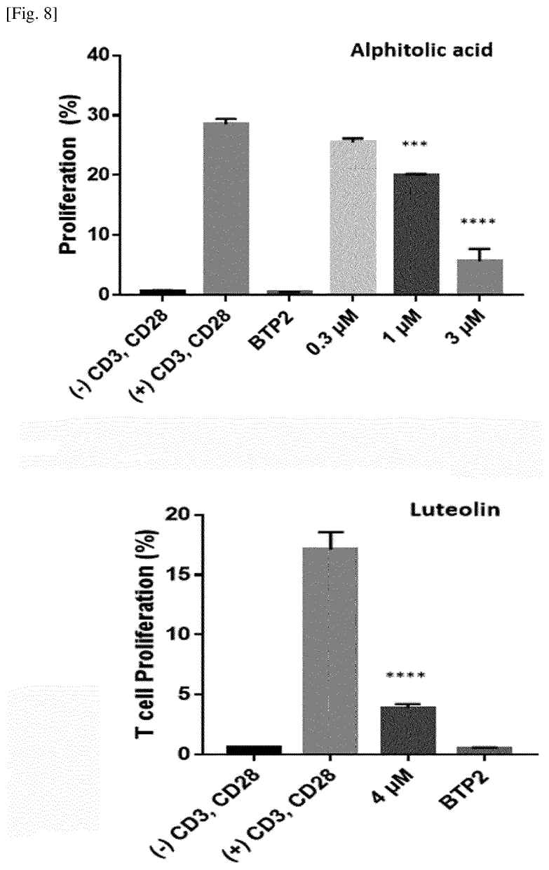

FIG. 9 represents the inhibitory effect of the inventive purified extract/comparative APH₂O/AP30%/AP70% extract on the release of cytokines;

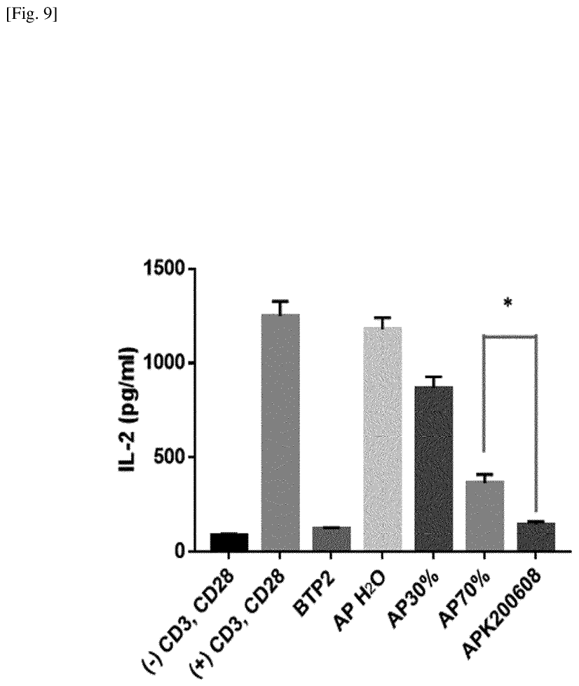

FIG. 10 represents the inhibitory effect of the inventive purified extract/comparative APH₂O/AP30%/AP70% extract on the degranulation of mast cell;

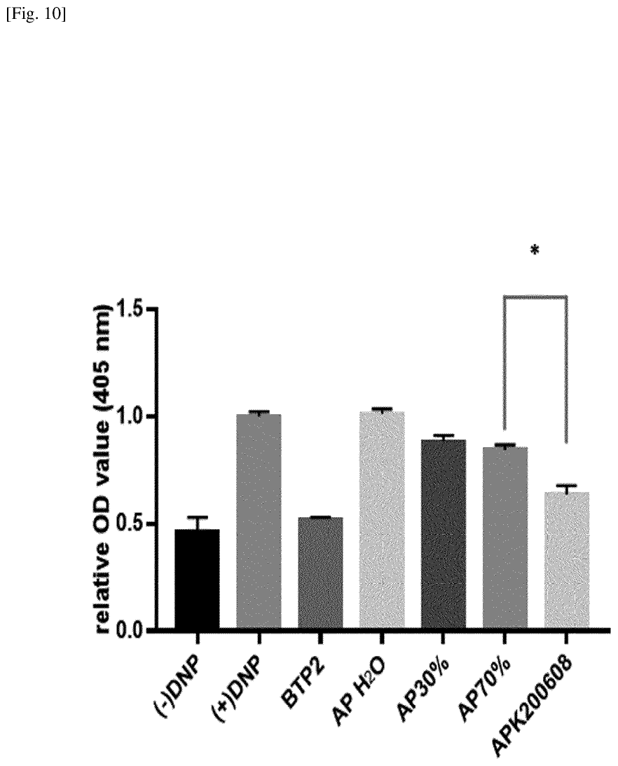

FIG. 11 presents the inhibitory effect of the inventive purified extract/comparative APH₂O/AP30%/AP70% extract on ORAI1.

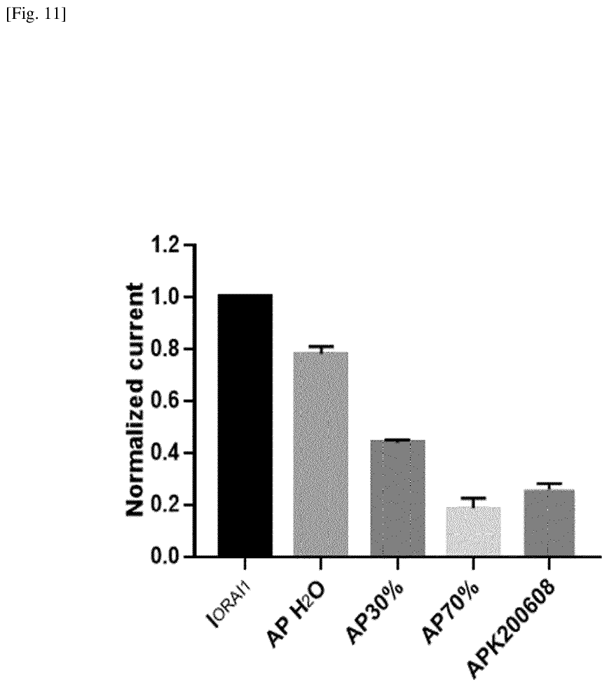

### MODE FOR THE INVENTION

Hereinafter, the formulating methods and kinds of excipients will be described, but the present invention is not limited to them. The representative preparation examples were described as follows.

### Preparation of Injection

APK200608 extract: 100 mg
Sodium metabisulfite: 3.0 mg
Methyl paraben: 0.8 mg
Propyl paraben: 0.1 mg
Distilled water for injection: optimum amount
Injection preparation was prepared by dissolving active component, controlling pH to about 7.5 and then filling all the components in 2 [symbol] ample and sterilizing by conventional injection preparation method.

### Preparation of Powder

APK200608 extract: 500 mg
Corn Starch: 100 mg
Lactose: 100 mg
Talc: 10 mg
Powder preparation was prepared by mixing above components and filling sealed package.

### Preparation of Tablet

APK200608 extract 200 mg
Corn Starch 100 mg
Lactose 100 mg
Magnesium stearate optimum amount
Tablet preparation was prepared by mixing above components and entabletting.

### Preparation of Capsule

APK200608 extract: 100 mg
Lactose: 50 mg
Corn starch: 50 mg
Talc: 2 mg
Magnesium stearate optimum amount
Tablet preparation was prepared by mixing above components and filling gelatin capsule by conventional gelatin preparation method.

### Preparation of Liquid

APK200608 extract: 1000 mg
Sugar: 20 g
Polysaccharide: 20 g
Lemon flavor: 20 g
Liquid preparation was prepared by dissolving active component, and then filling all the components in 1000 [symbol] ample and sterilizing by conventional liquid preparation method.

### Preparation of Health Food

APK200608 extract: 1000 mg
Vitamin mixture: optimum amount
Vitamin A acetate: 70 g
Vitamin E: 1.0 mg
Vitamin B₁₀: 13 mg
Vitamin B₂: 0.15 mg
Vitamin B6: 0.5 mg
Vitamin B1: 20.2 g
Vitamin C: 10 mg
Biotin: 10 g
Amide nicotinic acid: 1.7 mg
Folic acid: 50 g
Calcium pantothenic acid: 0.5 mg
Mineral mixture: optimum amount
Ferrous sulfate: 1.75 mg
Zinc oxide: 0.82 mg
Magnesium carbonate: 25.3 mg
Monopotassium phosphate: 15 mg
Dicalcium phosphate: 55 mg
Potassium citrate: 90 mg
Calcium carbonate: 100 mg
Magnesium chloride: 24.8 mg

The above mentioned vitamin and mineral mixture may be varied in many ways. Such variations are not to be regarded as a departure from the spirit and scope of the present invention.

### Preparation of Health Beverage

APK200608 extract: 1000 mg
Citric acid: 1000 mg
Oligosaccharide: 100 g
Apricot concentration: 2 g
Taurine: 1 g
Distilled water: 900

[symbol]

Health beverage preparation was prepared by dissolving active component, mixing, stirred at 85° C. for 1 hour, filtered and then filling all the components in 1000 [symbol] ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the present invention provides novel industrialized method for preparing purified extract (APK200608) from Agrimonia coreana NAKAI, a Korean native plant, which can provide more abundant active ingredients showing potent ant-inflammatory activity comparing with the crude extract prepared by the conventional method disclosed in the prior art or previous known extract of Agrimonia pilosa; as well as the inventive purified extract showed more potent anti-inflammatory, anti-allergy and anti-atopic activity than that prepared by the con-ventional preparation method through various tests such as inhibition effect on T cell proliferation (Experimental Example 1), inhibition effect on IL-2 release in Jurkat T cell (Experimental Example 2), inhibition effect on degranulation in mast cell (Experimental Example 3), inhibition effect on ORAI1 ion channel regulating calcium signaling in cell (Experimental Example 4), therefore, it is confirmed that inventive purified extract is very useful in the alleviation or treatment of inflammatory disease, allergic disease and atopic dermatitis disease.

1. Novel purified extract APK200608 from the extract of Agrimonia coreana NAKAI characterized by containing 0.5-1.5 weight part (w/w) luteolun 7-glucuronide, 0.5-1.8 weight part (w/w) apigenin 7-glucuronide, 0.005-0.015 weight part (w/w) luteolin, 0.0050-0.0090 weight part (w/w) apigenin, 0.0010-0.0030 weight part (w/w) kaempferol, and 0.0040-0.0098 weight part (w/w) quercetin based on the weight of total extract (100%) of Agrimonia coreana NAKAI.

2. Novel purified extract APK200608 from the extract of Agrimonia coreana NAKAI characterized by having the relative mixed weight ratio (w/w) between the weight of each flavonoid derivative, of 400.0-650.0 (w/w) luteolun 7-glucuronide, 550.0-750.0 (w/w) apigenin 7-glucuronide, 2.0-10.0 (w/w) luteolin, 1.5-7.0 (w/w) apigenin, 1 (w/w) kaempferol, and 1.5-7.5 (w/w) quercetin.

3-14. (canceled)

15. A method of treating or preventing inflammatory, allergic or atopic dermatitis disease in mammals, wherein the method comprises administering a therapeutically effective amount of purified extract APK200608 from the extract of Agrimonia coreana NAKAI as set forth in claim 1 into the mammal suffering from inflammatory, allergic or atopic dermatitis diseases.


SEQUENCE LISTING (14 sequences)

SEQ ID NO: 1 - protein, 25 aa, Artificial Sequence
  primers for PCR analysis
CGTACAGTACTTTTGTGTTTACGAG

SEQ ID NO: 2 - protein, 25 aa, Artificial Sequence
  primers for PCR analysis
CCCATTCATCTGGAAATCTTGGTTC

SEQ ID NO: 3 - protein, 20 aa, Artificial Sequence
  primers for PCR analysis
GTAAAATCAAGTCCACCGCG

SEQ ID NO: 4 - protein, 26 aa, Artificial Sequence
  primers for PCR analysis
ATGTCACCACAAACAGAGACTAAAGC

SEQ ID NO: 5 - protein, 20 aa, Artificial Sequence
  primers for PCR analysis
TCGCTTAACACTCCCCTTCC

SEQ ID NO: 6 - protein, 23 aa, Artificial Sequence
  primers for PCR analysis
GCTTTCATGGAAGCTTTAACAAT

SEQ ID NO: 7 - protein, 22 aa, Artificial Sequence
  primers for PCR analysis
GTTATGCATGAACGTAATGCTC

SEQ ID NO: 8 - protein, 23 aa, Artificial Sequence
  primers for PCR analysis
CGCGCGTGGTGGATTCACAATCC

SEQ ID NO: 9 - protein, 19 aa, Artificial Sequence
  primers for PCR analysis
TTAGCCTTTGTTTGGCAAG

SEQ ID NO: 10 - protein, 19 aa, Artificial Sequence
  primers for PCR analysis
ATAGTTTAAGAGTAAGCAT

SEQ ID NO: 11 - protein, 20 aa, Artificial Sequence
  primers for PCR analysis
CGAAATCGGTAGACGCTACG

SEQ ID NO: 12 - protein, 20 aa, Artificial Sequence
  primers for PCR analysis
GGGGATAGAGGGACTTGAAC

SEQ ID NO: 13 - protein, 20 aa, Artificial Sequence
  primers for PCR analysis
GGTTCAAGTCCCTCTATCCC

SEQ ID NO: 14 - protein, 20 aa, Artificial Sequence
  primers for PCR analysis
ATTTGAACTGGTGACACGAG